(19) United States
(12) Patent Application Publication
(10) Pub. No.: US 2021/0130286 A1
(43) Pub. Date: May 6, 2021

(54) CATALYTIC C-X-BOND METATHESIS THROUGH ARYLATION

(71) Applicant: STUDIENGESELLSCHAFT KOHLE MBH, Mülheim an der Ruhr (DE)

(72) Inventors: Zhong LIAN, Mülheim an der Ruhr (DE); Bill MORANDI, Mülheim an der Ruhr (DE); Benjamin N. BHAWAL, Mülheim an der Ruhr (DE); Peng YU, Mülheim an der Ruhr (DE); Tristan DELCAILLAU, Mülheim an der Ruhr (DE)

(73) Assignee: STUDIENGESELLSCHAFT KOHLE MBH, Mülheim an der Ruhr (DE)

(21) Appl. No.: 16/491,594

(22) PCT Filed: Mar. 4, 2018
(86) PCT No.: PCT/EP2018/055238

§ 371 Date: Sep. 6, 2019

(30) Foreign Application Priority Data
Mar. 7, 2017 (DE) 10 2017 203 746.6

(51) Int. Cl. C07C 319/14; C07C 321/28; C07D 319/06; C07C 323/20; C07F 7/18; C07C 323/09; C07F 7/08; C07C 323/36; C07D 277/74; C07D 213/70; C07D 241/18; C07J 31/00; C07C 321/22; C07D 417/06

(52) U.S. Cl. CPC C07C 319/14; C07C 2602/42; C07D 319/06; C07C 323/20; C07F 7/1804; C07F 7/1892; C07C 323/09; C07F 7/083; C07F 7/081; C07C 323/36; C07D 277/74; C07D 213/70; C07D 241/18; C07J 31/003; C07C 321/22; C07D 417/06; C07C 2601/14; C07C 2601/08; C07C 2603/74; C07C 321/28

(57) ABSTRACT

The present invention refers to a process for a catalytic aryl transfer to rearrange the backbone of aromatic C—X bonds.

This application is a 371 of PCT/EP2018/055238, filed Mar. 4, 2018, which claims foreign priority benefit under 35 U.S.C. § 119 of German Patent Application No. 10 2017 203 746.6, filed Mar. 7, 2017, the disclosures of which are incorporated herein by reference.

The present invention refers to a process for a catalytic aryl transfer to rearrange the backbone of aromatic C—X bonds.

The alkene metathesis reaction has had a transformative impact on chemistry by offering an alternative approach to olefin synthesis that is complementary in scope and reactivity to traditional olefination reactions, such as the Wittig reaction. Due to its versatility, alkene metathesis has consequently found applications in very diverse areas, including polymer chemistry, biomass valorization and drug synthesis due to the ubiquity of alkenes as starting materials, synthetic intermediates and final products. The isodesmic nature of the reaction enables the transformation of one alkene into another in a mild process, leading to an overall exchange of the alkene group substituents (FIG. 1A). This feature facilitates the rapid generation of new molecular architectures while conserving the important olefin functionality. In light of the established synthetic power of alkene metathesis, it can be expected that the metathesis of other important bonds including single bonds would have a beneficial impact on the molecular sciences.

Carbon-heteroatom bonds composed of heavy main group elements are commonly encountered in a wide range of applications (FIG. 1B). In particular, C(sp²)-S and C(sp²)-P bonds are essential in materials and medicinal sciences. Aromatic thioethers are key motifs in drug development and can also be found in many organic materials and polymers—for example, the thermoplastic polyphenylene sulfide (PPS, 1) is produced yearly on a 80,000 ton scale. Aromatic phosphines are commonly used as ligands and catalysts, both on laboratory scale and industrial processes. They are further employed in the area of organic materials, with applications ranging from sensors to organic light emitting diodes.

There are rare examples of single C—X bond metathesis only, including transamidation processes. The metathesis of aromatic C—X bonds, however, has been virtually unexplored.

It is an object of the present invention therefore to provide a carbon-heteroatom bond metathesis reactions, wherein compounds containing at least one C(sp²)-heteroatom bond could effectively swap their substituents in a manner analogous to alkene metathesis. The challenge in developing catalytic metathesis reactions employing C—X bonds is to identify a mechanistic pathway in which the breakage of a C—X bond and subsequent ligand exchange can be realized. The reaction conditions wherein the oxidative addition and reductive elimination of common C—X bonds, including C—S or C—P bonds, and exchange of the resulting thiolate or phosphine ligand could be achieved to unlock novel catalytic C—X bond metathesis reactions should be identified. It is also an object to identify a general mechanistic manifold, proceeding through transfer arylation, to perform unprecedented catalytic C(sp²)-X bond metathesis with several different main group elements (FIG. 1C).

It has now been found that the above-mentioned disadvantages can be dealt with by a process for a catalytic aryl transfer wherein an aryl-compound (I) is reacted with an hydrocarbon (II) in the presence of a Pd- or Ni-catalyst coordinated by electron rich ligands and in the presence of a base in an organic solvent, as represented in the following reaction scheme:

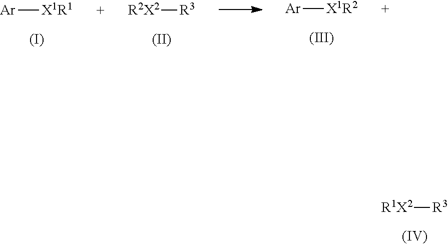

wherein
Ar is aryl, heteroaryl or vinyl, each being optionally substituted by one or more groups selected from straight chain or branched chain alkyl, cycloalkyl, heterocycloalkyl, alkenyl, alkynyl, aryl, aralkyl, heteroaryl, heteroaralkyl, ether, acetal, silyl ether or amine, or by a heterosubstituent,
X¹ and X² may be the same or different and are each S or Se, preferably S,
R¹ is H or methyl, a straight chain or branched C₂-C₁₆-alkyl, or aryl, each optionally being substituted by one or more groups selected from straight chain or branched chain alkyl, cycloalkyl, heterocycloalkyl, alkenyl, alkynyl, aryl, aralkyl, heteroaryl, heteroaralkyl, ether, acetal, silyl ether or amine, or by a heterosubstituent;
R² is an primary, secondary or tertiary alkyl or aryl, each being optionally being substituted by one or more groups selected from straight chain or branched chain alkyl, cycloalkyl, heterocycloalkyl, alkenyl, alkynyl, aryl, aralkyl, heteroaryl, heteroaralkyl, or by a heterosubstituent;
R³ is H,
the Pd- or Ni-catalyst coordinated by electron rich ligands is selected from the group consisting of Pd(OAc)₂, Pd₂(dba)₃, PdCl₂, PdCl₂(MeCN)₂, Ni(COD)₂.
the electron rich ligands is selected from the group consisting of IPENT(1,3-Bis(2,6-bis(1-ethylpropyl)phenyl)imidazol-2-ylidene), SIPr (1,3-Bis(2,6-diisopropylphenyl)imidazolidene), ICy (1,3-bis-(cyclohexyl)imidazol-2-ylidene), or IPr (1,3-bis(2,6-diisopropylphenyl)imidazol-2-ylidene).
the base is selected from the group consisting of LiHMDS, KHMDS, NaHDMS, LiOtBu, KOtBu, NaOtBu.

Such a process represents a powerful companion to traditional cross-coupling processes. This strategy is particularly useful for the rapid discovery and derivatization of functional molecules to prepare compound libraries that are essential for structure activity relationship (SAR) studies. Additionally, the process parallel the alkene metathesis reaction in providing an extremely flexible tool for the construction and deconstruction of organic molecules with potential applications in waste recycling and polymerization.

A further attractive feature of this reaction is the possibility, at higher temperatures, to directly use, for example, an unprotected thiophenol as an electrophile through C—S bond cleavage (FIG. 2B). This is notable, since previous examples of such reactivity are extremely rare due to catalyst poisoning, and only two thiophenol substrates have been reported to undergo C—S bond cleavage, using Grignard reagents under Ni-catalysis. Homodimerization of thiophenol, which could potentially have plagued the efficiency of a chemoselective coupling process, does not interfere with the inventive reaction when a more nucleophilic alkyl thiol is employed. However, if no cross-metathesis partner is present in the reaction mixture, homodimerization of thiophenol leads to the formation of diphenyl sulfide (44). Another chalcogen element, selenium, can also participate in this reaction and the homodimerization of selenophenol gave high yields of the corresponding diphenylselenide (45).

In one embodiment, a Pd—NHC (N-heterocyclic carbene) complex, preferably a [(NHC)Pd(dimethylbenzylamine)Cl] complex is used as catalyst. Such a catalyst efficiently promotes the oxidative addition of Ar—X and its microscopic reverse, reductive elimination, to enable a facile X—R³ group exchange to take place. According to the present invention, the Pd- or Ni-catalyst is present in an amount in the range of 0.05 mol % to 1 mol % of the aryl-compound (I), preferably in the range of 0.2 mol % to 0.6 mol % of the aryl-compound (I).

In a preferred embodiment, the base used in the process of the present invention is a lithium base, sodium base or potassium base, preferably a lithium base, more preferably LiHMDS (lithium bis(trimethylsilyl)amide). Use of a lithium base proved clearly superior to sodium or potassium bases, reflecting the importance of solubilities in this reaction. The decreased solubility of the side-product, for example MeSLi when Ar—XR¹ is a methyl aromatic thioether, when compared to the larger lithium thiolate salt generated from the thiol reagent and the base, efficiently drives the equilibrium of the reaction to completion.

Preferably, the base is present in an amount in the range of 1 equivalent to 6 equivalents, preferably in the range of 1.5 equivalent to 4 equivalents of the reaction partners.

In a preferred embodiment, the aryl-compound (I) is reacted with the hydrocarbon (II) at a temperature in the range of 25° C. to 250° C., preferably in the range of 80° C. to 200° C., for 4 h to 20 h, preferably 8 h to 16 h.

The organic solvent used for reacting the aryl-compound (1) with the hydrocarbon (II) is not critical and can be selected amongst those which are commonly used for such kind of catalyzed reactions. Preferably, an aromatic solvent or an aliphatic hydrocarbon solvent, more preferably toluene, benzene, xylene, cumene, chlorobenzene or dichlorobenzene is used.

In a preferred embodiment, Ar is phenyl, 4-methylphenyl or naphtyl, each optionally being substituted by one or more groups selected from straight chain or branched chain alkyl, ether, acetal, silyl ether or amine, preferably being substituted by methyl, ketals, methoxy, MOMO, TIPSO, OCF₃, OBn, CF₃, F, TMS, NMe₂, CN, pyridyl, pyrazinyl, benzothiazyl, phenylvinyl, t-butyl or 5-phenyl-benzothiazyl.

In a preferred embodiment, Ar or Ar—X is a component of a polymer. The process of the present invention provides the possibility to depolymerize polymers, preferably, thermoplastic polymers, in particular PPS (1) to obtain simple chemical building blocks (FIG. 1B).

In a preferred embodiment, X is S.

In a further embodiment, R¹ is H, methyl, phenyl or 4-methoxyphenyl.

In one embodiment, R² is cyclohexyl, cyclopentyl, 2-methylbutyl, 1-methyl-propyl, nC₁₂H₂₅, adamantyl, 2-phenylethyl, 1-methyl-5-dimethyl-bicyclo[4.1.0]heptyl-, nC₈H₁₇, benzyl, optionally substituted steroid residue, 2-amantadyl-ethyl or C₈H₁₇.

In a preferred embodiment, the hydrocarbon (II) is present an amount in the range of 0.5 equivalents to 6 equivalents of the aryl-compound (1), preferably in the range of 1.5 equivalent to 4 equivalents of the aryl-compound (1).

In a further embodiment, the process of the present invention is used to enable a sequence of arylation/retro-arylation to take place and equilibrate a simple reaction mixture (FIG. 1C). Such a thioether cross-metathesis, in case X is S, leads to the same ratio of starting materials to products in the forward and reverse direction. In such an process, R²X—R³ is used as a co-catalytic amount in the range of 5 to 15%, preferably in the range of 7.5 to 12.5% of the total amount of the at least one Ar—X—R¹ compound.

Accordingly, the present invention also provides an aryl-compound produced by the process of the present invention.

Definition for the substituents as described herein are given in the following.

A heterosubstituent according to the invention is to be understood as a substituent including heteroatoms, preferentially selected from O, N, S, Si and halogens. It can be preferentially selected from, ═O, —OH, —F, —Cl, —Br, —I, —CN, —N₃, —NO₂, —SO₃H, NCO, NCS, OP(O)(OR^{S1})(OR^{S2}), OP(OR^{S1})(OR^{S2}), a monohalogenomethyl group, a dihalogenomethyl group, a trihalogenomethyl group, —CF(CF₃)₂, —SF₅, —NRS₂, —OR^{S1}, —OOR^{S1}, —OSiR^{S1}R^{S2}R^{S3}, —OSi(OR^{S1})R^{S2}R^{S3}, —OSi(OR^{S1})(OR^{S2})R^{S3}, —OSi(OR^{S1})(OR^{S2})(OR^{S3}), —OSO₂R^{S1}, —SR^{S1}, —SSR^{S1}, —S(O)R^{S1}, —S(O)₂R^{S1}, —C(O)OR^{S1}, —C(O)NR^{S1}R^{S2}, —NR^{S1}C(O)R^{S2}, —C(O)—R^{S1}, —COOM, wherein M may be a metal such as Na, K or Cs.

R^{S1} R^{S2} and R^{S3} each individually represent H, alkyl, cycloalkyl, heterocycloalkyl, aryl, heteroaryl, aralkyl, heteroaralkyl, sulfonyl, silyl, each being optionally substituted by one or more alkyl, cycloalkyl, heterocycloalkyl, heteroaryl, aralkyl, heteroaralkyl, sulfonyl or heterosubstituent.

For the reaction system in more detail, alkyl may be C1-C20-Alkyl which can be straight chain or branched or cyclic and has 1, 2, 3, 4, 5, 6, 7, 8, 9, 10, 11, 12, 13, 14, 15, 16, 17, 18, 19 or 20 carbon atoms. Alkyl might particularly be C1-C6-alkyl, in particular methyl, ethyl, propyl, isopropyl, butyl, isobutyl, sec-butyl or tert-butyl, likewise pentyl, 1-, 2- or 3-methylpropyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl.

Cycloalkyl may be a cyclic alkyl group forming a 3 to 20 membered ring and might be cyclopropyl, cyclobutyl, cyclopentyl, cyclohexyl, cycloheptyl or cyclooctyl.

Heterocycloalkyl may be a cycloalkyl forming a 3 to 10 membered ring and incorporating one or more heteroatoms selected from N, O and S within the cycle. In particular, heterocycloalkyls can be preferentially selected from 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl.

Halogen is F, Cl, Br or I.

Aryl might be phenyl, naphthyl or biphenyl and substituted derivatives thereof.

Aralkyl might be benzyl, naphthylmethyl and substituted derivatives thereof.

Heteroaryl may have one or more heteroatoms selected from N, O, S and Si and is preferably 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, also preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-Indolyl, 4- or 5-isoindolyl, 1-, 2-, 4- or 5-benz-imidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, also preferably 1,3-benzodioxol-5-yl, 1,4-benzodioxan-6-yl, 2,1,3-benzothiadiazol-4- or -5-yl or 2,1,3-benzoxadiazol-5-yl.

Heteroaralkyl might be any of the aforementioned heteroaryl bound to an alkyl group, such as pyridinylmethyl.

Optionally substituted means unsubstituted or monosubstituted, disubstituted, trisubstituted, tetrasubstituted, pentasubstituted, or even further substituted on the respective group.

### BRIEF DESCRIPTION OF THE DRAWINGS

The invention is further illustrated in the attached figures and the following experimental section below.

In the attached drawings:

FIG. 1A illustrates alkene metathesis.

FIG. 1B illustrates selected applications of heavy main group elements bound to C(sp²).

FIG. 1C illustrates the process of the present invention showing a single-bond metathesis through arylation.

FIG. 2A illustrates products obtained by the process of the present invention using aromatic thioethers as Ar—XR¹ educt.

FIG. 2B illustrates products obtained by the process of the present invention using thiophenols as Ar—XR¹ educt.

FIG. 3A and FIG. 3B illustrate the synthetic potential of the C—S bond metathesis reaction of the present invention. In particular:

FIG. 3A shows late stage generation of a drug library using Thioridazine as Ar—XR¹ starting material.

FIG. 3B shows depolymerization of a commercial plastic.

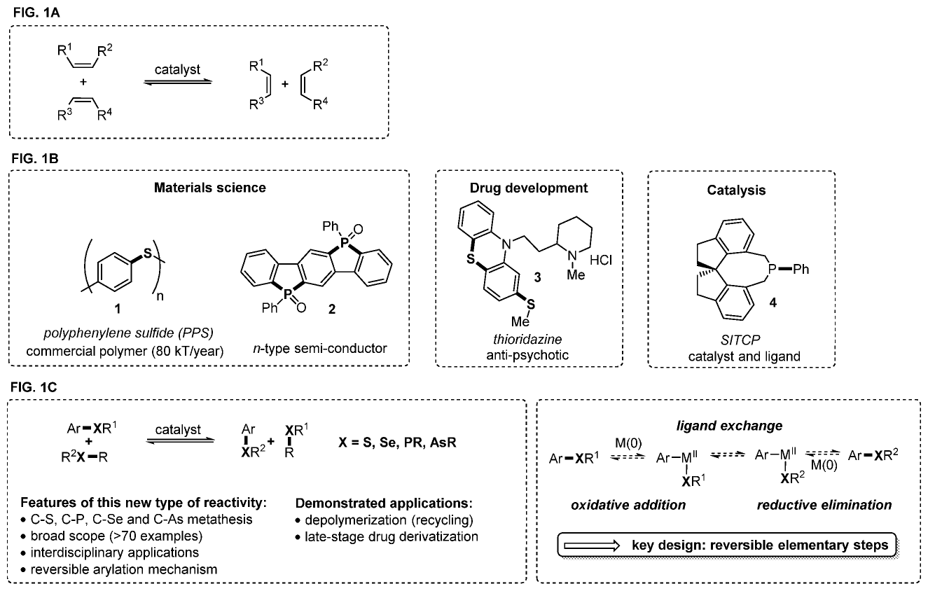

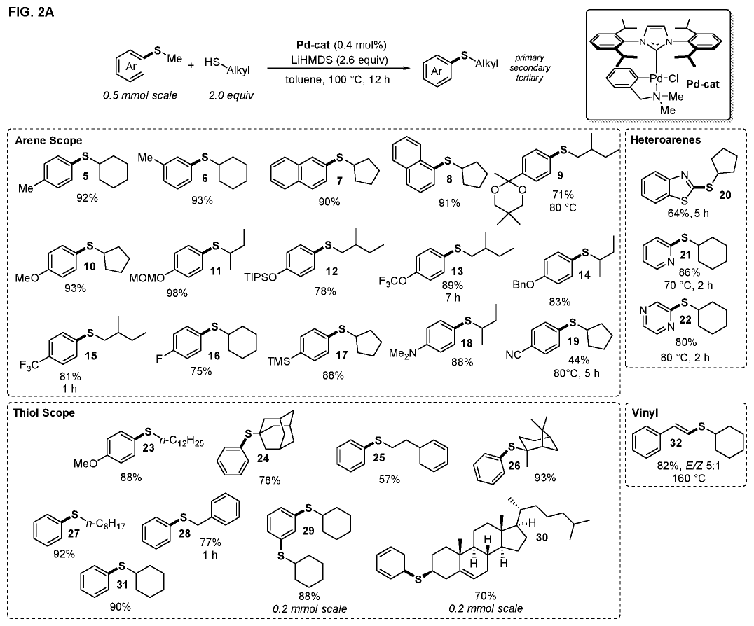

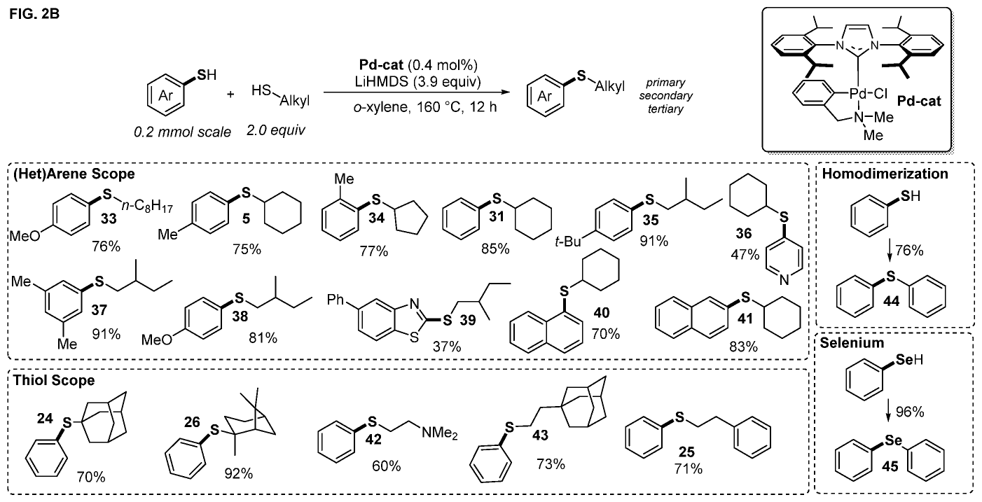

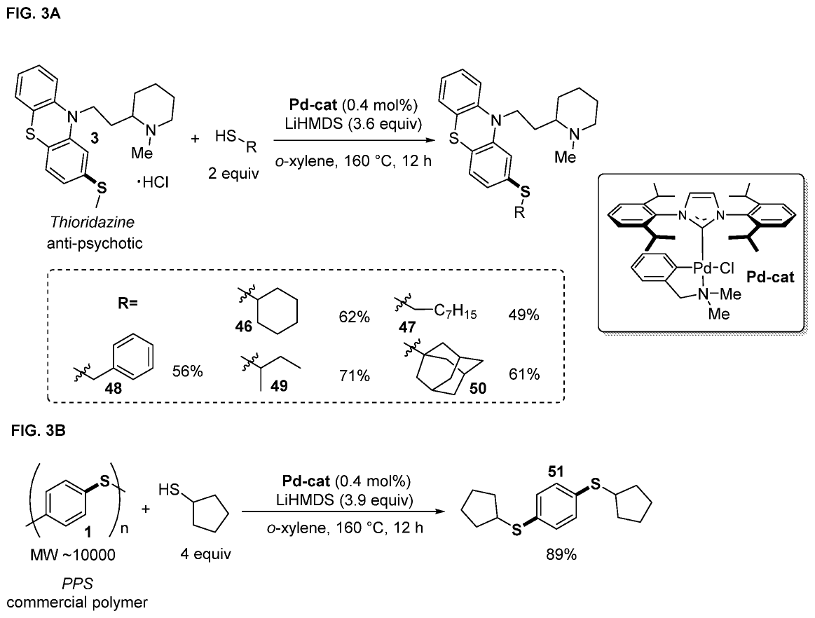

### EXPERIMENTAL SECTION

### Preparation Examples

General Procedure for the Catalytic Aryl Transfer with Aryl Ethers

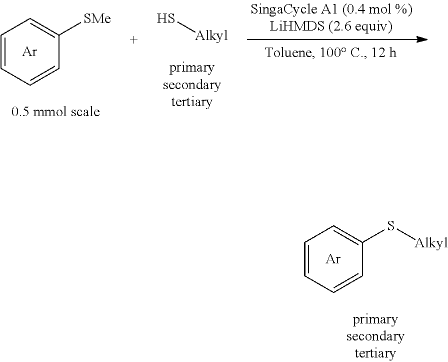

In the glovebox, aryl methyl sulfane (0.5 mmol), alkyl thiol (2.0 equiv, 1.0 mmol), LiHMDS (1.3 ml, 1.0 M in toluene), and SingaCycle A1 (0.4 mol %, 0.4 ml, 0.005 M in toluene) were added into an oven-dried 8 ml vial with a magnetic stirring bar, followed by addition of toluene (0.3 ml). The vial was sealed and removed out of the glovebox and heated to 100° C. After 12 h, the vial was cooled to room temperature. The reaction was diluted with ethyl acetate and washed with saturated NaOH solution. The aqueous phase was extracted with ethyl acetate 3 times. The collected organic phases were dried over anhydrous Na₂SO₄. The solvent was removed under reduced pressure and the residue was purified by flash column chromatography to give the desired product.

### Example 1

### Cyclohexyl(p-Tolyl)Sulfane

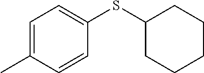

Prepared by general procedure A; isolated as a colorless liquid using pentane/ethyl acetate (100:1) as eluent (94.2 mg, 92%). ¹H NMR (300 MHz, Chloroform-d) δ 7.31 (d, J=7.8 Hz, 2H), 7.10 (d, J=7.8 Hz, 2H), 3.12-2.94 (m, 1H), 2.33 (s, 3H), 2.06-1.91 (m, 2H), 1.84-1.71 (m, 2H), 1.66-1.57 (m, 1H), 1.44-1.15 (m, 5H). ¹³C NMR (75 MHz, CDCl₃) δ 136.8, 132.7, 131.2, 129.5, 47.1, 33.4, 26.1, 25.8, 21.0. HRMS C₁₃H₁₈S [M]⁺; calculated 206.1123, found: 206.1126. The spectral data are consistent with those reported in the literature.

### Example 2

### Cyclohexyl(m-Tolyl)Sulfane

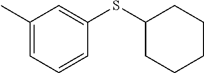

Prepared by general procedure A; isolated as a colorless liquid using pentane/ethyl acetate (100:1) as eluent (95 mg, 93%). ¹H NMR (300 MHz, Chloroform-d) δ 7.25-7.12 (m, 3H), 7.03 (s, 1H), 3.23-3.01 (m, 1H), 2.33 (s, 3H), 2.05-1.93 (m, 2H), 1.77 (q, J=5.4, 4.4 Hz, 2H), 1.66-1.58 (m, 1H), 1.45-1.20 (m, 5H). ¹³C NMR (126 MHz, CDCl₃) δ 138.5, 134.9, 132.5, 128.8, 128.6, 127.5, 46.6, 33.4, 26.1, 25.8, 21.3. HRMS C₁₃H₁₈S [M]⁺; calculated 206.1123, found: 206.1125. The spectral data are consistent with those reported in the literature.

### Example 3

### Cyclopentyl(naphthalen-2-yl)sulfane

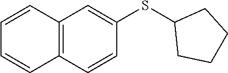 [SMILES: c1ccc2cc(SC3CCCC3)ccc2c1]

Prepared by general procedure A; isolated as a pale yellow liquid using pentane/ethyl acetate (100:1) as eluent (102.3 mg, 90%). ¹H NMR (300 MHz, Chloroform-d) δ 7.87-7.73 (m, 4H), 7.57-7.38 (m, 3H), 3.96-3.65 (m, 1H), 2.24-2.03 (m, 2H), 1.92-1.60 (m, 6H). ¹³C NMR (75 MHz, CDCl₃) δ 134.8, 133.7, 131.7, 128.1, 128.0, 127.64, 127.63, 127.0, 126.4, 125.5, 45.8, 33.5, 24.8. HRMS C₁₅H₁₆S [M]⁺; calculated 228.0967, found: 228.0968.

### Example 4

### Cyclopentyl(naphthalen-1-yl)sulfane

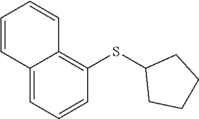 [SMILES: c1ccc2c(SC3CCCC3)cccc2c1]

Prepared by general procedure A; isolated as a pale yellow liquid using pentane/ethyl acetate (100:1) as eluent (103 mg, 91%). ¹H NMR (300 MHz, Chloroform-d) δ 8.55-8.42 (m, 1H), 7.92-7.82 (m, 1H), 7.81-7.71 (m, 1H), 7.65 (dd, J=7.2, 1.2 Hz, 1H), 7.58-7.52 (m, 2H), 7.43 (dd, J=8.2, 7.2 Hz, 1H), 3.68 (td, J=6.3, 5.7, 1.4 Hz, 1H), 2.19-1.96 (m, 2H), 1.92-1.80 (m, 2H), 1.78-1.58 (m, 4H). ¹³C NMR (75 MHz, CDCl₃) δ 134.3, 133.9, 133.3, 129.2, 128.4, 127.2, 126.2, 126.1, 125.5, 125.3, 46.5, 33.6, 24.7. HRMS C₁₅H₁₆S [M]⁺; calculated 228.0967, found: 228.0969.

### Example 5

### 2,5,5-trimethyl-2-(4-((2-methylbutyl)thio)phenyl)-1,3-dioxane

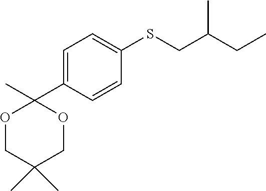 [SMILES: CCC(C)CSc1ccc(C2(C)OCC(C)(C)CO2)cc1]

Prepared by general procedure A at 80° C.; isolated as a yellow liquid using pentane/ethyl acetate (30:1) as eluent (109 mg, 71%). ¹H NMR (500 MHz, Chloroform-d) δ 7.36-7.27 (m, 4H), 3.45-3.32 (m, 4H), 2.97 (dd, J=12.4, 5.8 Hz, 1H), 2.78 (dd, J=12.4, 7.4 Hz, 1H), 1.79-1.64 (m, 1H), 1.60-1.53 (m, 1H), 1.51 (s, 3H), 1.34-1.27 (m, 1H), 1.26 (s, 3H), 1.04 (d, J=6.7 Hz, 3H), 0.92 (t, J=7.4 Hz, 3H), 0.58 (s, 3H). ¹³C NMR (126 MHz, CDCl₃) δ 138.1, 137.1, 128.4, 127.3, 100.0, 71.7, 40.4, 34.5, 31.9, 29.9, 28.8, 22.9, 21.9, 19.0, 11.3. HRMS C₁₈H₂₈O₂S [M]⁺; calculated 308.1810, found: 308.1809.

### Example 6

### Cyclopentyl(4-methoxyphenyl)sulfane

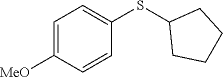 [SMILES: COc1ccc(SC2CCCC2)cc1]

Prepared by general procedure A; isolated as a colorless liquid using pentane/ethyl acetate (50:1) as eluent (95.9 mg, 93%). ¹H NMR (500 MHz, Chloroform-d) δ 7.37 (d, J=8.8 Hz, 2H), 6.84 (d, J=8.8 Hz, 2H), 3.80 (s, 3H), 3.48-3.38 (m, 1H), 2.01-1.89 (m, 2H), 1.81-1.73 (m, 2H), 1.63-1.50 (m, 4H). ¹³C NMR (126 MHz, CDCl₃) δ 159.0, 134.1, 127.0, 114.4, 55.3, 48.0, 33.4, 24.6. HRMS C₁₂H₁₆SO [M]⁺; calculated 208.0916, found: 208.0919. The spectral data are consistent with those reported in the literature.

### Example 7

### Sec-butyl(4-(methoxymethoxy)phenyl)sulfane

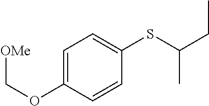

Prepared by general procedure A; isolated as a colorless liquid using pentane/ethyl acetate (30:1) as eluent (110.3 mg, 98%). ¹H NMR (300 MHz, Chloroform-d) δ 7.43-7.32 (m, 2H), 7.03-6.87 (m, 2H), 5.16 (s, 2H), 3.48 (s, 3H), 2.99 (td, J=6.9, 6.1 Hz, 1H), 1.62-1.38 (m, 2H), 1.23 (d, J=6.7 Hz, 3H), 0.99 (t, J=7.4 Hz, 3H). ¹³C NMR (75 MHz, Chloroform-d) δ 156.8, 135.2, 126.9, 116.6, 94.4, 56.0, 46.1, 29.4, 20.5, 11.5. HRMS C₁₂H₁₈O₂S [M+H]⁺; calculated 227.1100, found: 227.1102.

### Example 8

### Triisopropyl(4-((2-methylbutyl)thio)phenoxy)silane

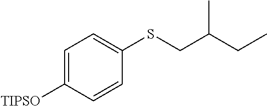

Prepared by general procedure A; isolated as a colorless liquid using pentane/ethyl acetate (50:1) as eluent (136.1 mg, 78%). ¹H NMR (300 MHz, Chloroform-d) δ 7.35-7.19 (m, 2H), 6.89-6.76 (m, 2H), 2.87 (dd, J=12.6, 5.7 Hz, 1H), 2.68 (dd, J=12.6, 7.4 Hz, 1H), 1.72-1.45 (m, 2H), 1.26 (dd, J=14.8, 6.8 Hz, 4H), 1.19-1.06 (m, 18H), 1.01 (d, J=6.6 Hz, 3H), 0.89 (t, J=7.4 Hz, 3H). ¹³C NMR (75 MHz, CDCl₃) δ 155.0, 132.3, 128.0, 120.4, 42.8, 34.6, 28.7, 18.8, 17.9, 12.6, 11.2. HRMS C₂₀H₃₆OSSi [M]⁺; calculated 352.2251, found: 352.2249.

### Example 9

### (2-methylbutyl)(4-(trifluoromethoxy)phenyl)sulfane

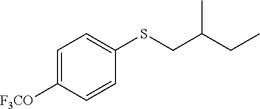 [SMILES: CCC(C)CSc1ccc(OC(F)(F)F)cc1]

Prepared by general procedure A for 7 h; isolated as a pale yellow liquid using pentane/ethyl acetate (50:1) as eluent (117 mg, 89%). ¹H NMR (300 MHz, Chloroform-d) δ 7.38-7.31 (m, 2H), 7.19-7.11 (m, 2H), 2.96 (dd, J=12.5, 5.8 Hz, 1H), 2.77 (dd, J=12.5, 7.4 Hz, 1H), 1.76-1.48 (m, 2H), 1.37-1.23 (m, 1H), 1.05 (d, J=6.6 Hz, 3H), 0.93 (t, J=7.4 Hz, 3H). ¹⁹F NMR (282 MHz, Chloroform-d) δ −58.0. ¹³C NMR (75 MHz, Chloroform-d) δ 147.1 (q, J=1.9 Hz), 136.4, 129.9, 121.4, 120.4 (q, J=257.0 Hz), 41.0, 34.4, 28.7, 18.9, 11.2. HRMS C₁₂H₁₅SOF₃ [M]⁺; calculated 264.0790, found: 264.0792.

### Example 10

### (4-(benzyloxy)phenyl)(sec-butyl)sulfane

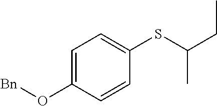 [SMILES: CCC(C)Sc1ccc(OCc2ccccc2)cc1]

Prepared by general procedure A; isolated as a pale yellow liquid using pentane/ethyl acetate (50:1) as eluent (113.3 mg, 83%). ¹H NMR (300 MHz, Chloroform-d) δ 7.48-7.30 (m, 7H), 6.92 (d, J=8.8 Hz, 2H), 5.05 (s, 2H), 2.97 (td, J=6.9, 6.1 Hz, 1H), 1.70-1.39 (m, 2H), 1.22 (d, J=6.8 Hz, 3H), 1.00 (t, J=7.4 Hz, 3H). ¹³C NMR (75 MHz, Chloroform-d) δ 158.5, 136.8, 135.5, 128.6, 128.0, 127.5, 125.6, 115.2, 70.1, 46.2, 29.4, 20.5, 11.5. HRMS C₁₇H₂₀OS [M]⁺; calculated 272.1229, found: 272.1232.

### Example 11

### (2-methylbutyl)(4-(trifluoromethyl)phenyl)sulfane

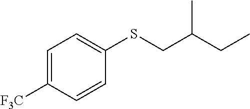 [SMILES: CCC(C)CSc1ccc(C(F)(F)F)cc1]

Prepared by general procedure A for 1 h; isolated as a pale yellow liquid using pentane/ethyl acetate (80:1) as eluent (100.1 mg, 81%). ¹H NMR (300 MHz, Chloroform-d) δ 7.52 (dt, J=8.3, 0.7 Hz, 2H), 7.41-7.34 (m, 2H), 3.01 (dd, J=12.5, 5.8 Hz, 1H), 2.82 (dd, J=12.5, 7.5 Hz, 1H), 1.79-1.69 (m, 1H), 1.62-1.51 (m, 1H), 1.34 (dd, J=14.3, 6.7 Hz, 1H), 1.07 (d, J=6.6 Hz, 3H), 0.95 (t, J=7.4 Hz, 3H). ¹⁹F NMR (282 MHz, Chloroform-d) δ −62.4. ¹³C NMR (126 MHz, Chloroform-d) δ 143.2, 127.1, 127.0 (q, J=32.4 Hz), 125.5 (q, J=3.8 Hz), 124.2 (q, J=271.6 Hz), 39.5, 34.4, 28.8, 19.0, 11.2. HRMS C₁₂H₁₅SF₃ [M]⁺; calculated 248.0841, found: 248.0843.

### Example 12

### Cyclohexyl(4-fluorophenyl)sulfane

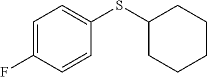 [SMILES: Fc1ccc(SC2CCCCC2)cc1]

Prepared by general procedure A; isolated as a colorless liquid using pentane/ethyl acetate (100:1) as eluent (78.5 mg, 75%). ¹H NMR (300 MHz, Chloroform-d) δ 7.42 (dd, J=8.8, 5.3 Hz, 2H), 7.01 (t, J=8.7 Hz, 2H), 3.00 (ddt, J=10.3, 7.2, 3.8 Hz, 1H), 1.99-1.90 (m, 2H), 1.85-1.71 (m, 2H), 1.66-1.56 (m, 1H), 1.44-1.09 (m, 5H). ¹⁹F NMR (282 MHz, Chloroform-d) δ −114.9. ¹³C NMR (75 MHz, Chloroform-d) δ 162.2 (d, J=246.8 Hz), 134.9 (d, J=8.1 Hz), 129.8 (d, J=3.4 Hz), 115.7 (d, J=21.6 Hz), 47.5, 33.3, 26.0, 25.7. HRMS C₁₂H₁₅FS [M]⁺; calculated 210.0873, found: 210.0872. The spectral data are consistent with those reported in the literature.

### Example 13

### (4-(cyclopentylthio)phenyl)trimethylsilane

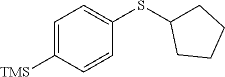 [SMILES: C[Si](C)(C)c1ccc(SC2CCCC2)cc1]

Prepared by general procedure A; isolated as a colorless liquid using pentane as eluent (109.7 mg, 88%). ¹H NMR (300 MHz, Chloroform-d) δ 7.42 (d, J=8.2 Hz, 2H), 7.32 (d, J=8.2 Hz, 2H), 3.62 (ddt, J=7.0, 3.4, 1.6 Hz, 1H), 2.23-2.00 (m, 2H), 1.88-1.72 (m, 2H), 1.72-1.56 (m, 4H), 0.25 (s, 9H). ¹³C NMR (126 MHz, CDCl₃) δ 139.5, 138.4, 134.8, 129.6, 46.4, 34.7, 26.0, 0.0. HRMS C₁₄H₂₂SSi [M]⁺; calculated 250.1206, found: 250.1204.

### Example 14

### 4-(sec-butylthio)-N,N-dimethylaniline

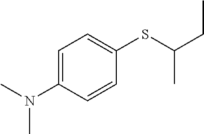

Prepared by general procedure A; isolated as a pale yellow liquid using pentane/ethyl acetate (50:1) as eluent (91.8 mg, 88%). ¹H NMR (300 MHz, Chloroform-d) δ 7.41-7.30 (m, 2H), 6.79-6.55 (m, 2H), 2.96 (s, 6H), 2.89 (q, J=6.7 Hz, 1H), 1.69-1.37 (m, 2H), 1.21 (d, J=6.7 Hz, 3H), 0.99 (t, J=7.4 Hz, 3H). ¹³C NMR (126 MHz, Chloroform-d) δ 136.0 (2C), 112.6 (2C), 46.4, 40.5, 29.4, 20.5, 11.6. HRMS C₁₂H₁₉NS [M+H]⁺; calculated 210.1311, found: 210.1308.

### Example 15

### 4-(cyclopentylthio)benzonitrile

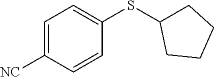 [SMILES: N#Cc1ccc(SC2CCCC2)cc1]

Prepared by general procedure A for 5 h at 80° C.; isolated as a pale yellow liquid using pentane/ethyl acetate (50:1) as eluent (49 mg, 44%). ¹H NMR (300 MHz, Chloroform-d) δ 7.49-7.39 (m, 2H), 7.28-7.21 (m, 2H), 3.74-3.53 (m, 1H), 2.08 (dddd, J=9.4, 4.8, 2.5, 1.0 Hz, 2H), 1.73 (d, J=1.4 Hz, 2H), 1.66-1.47 (m, 4H). ¹³C NMR (75 MHz, Chloroform-d) δ 145.8, 132.0, 127.2, 118.9, 107.7, 44.0, 33.3, 24.9. HRMS C₁₂H₁₃NS [M+Na]⁺; calculated 226.0661, found: 226.0661.

### Example 16

### 2-(cyclopentylthio)benzo[d]thiazole

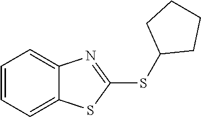 [SMILES: c1ccc2sc(SC3CCCC3)nc2c1]

Prepared by general procedure A for 5 h; isolated as a yellow liquid using pentane/ethyl acetate (30:1) as eluent (75.2 mg, 64%). ¹H NMR (500 MHz, Chloroform-d) δ 7.88 (dd, J=8.2, 1.0 Hz, 1H), 7.76 (dd, J=7.9, 1.1 Hz, 1H), 7.41 (ddd, J=8.3, 7.2, 1.2 Hz, 1H), 7.32-7.27 (m, 1H), 4.20-4.07 (m, 1H), 2.34-2.23 (m, 2H), 1.90-1.64 (m, 6H). ¹³C NMR (75 MHz, Chloroform-d) δ 167.5, 153.4, 135.2, 126.0, 124.1, 121.5, 120.8, 46.7, 33.8, 24.9. HRMS C₁₂H₁₃NS₂ [M+Na]⁺; calculated 258.0382, found: 258.0379. The spectral data are consistent with those reported in the literature.

### Example 17

### 2-(cyclohexylthio)pyridine

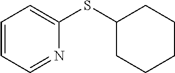 [SMILES: c1ccc(SC2CCCCC2)nc1]

Prepared by general procedure A for 2 h at 70° C.; isolated as a pale yellow liquid using pentane/ethyl acetate (20:1) as eluent (83 mg, 86%). ¹H NMR (300 MHz, Chloroform-d) δ 8.42 (dt, J=4.8, 1.5 Hz, 1H), 7.45 (ddd, J=8.0, 7.3, 1.9 Hz, 1H), 7.18-7.12 (m, 1H), 6.95 (ddd, J=7.4, 4.9, 1.1 Hz, 1H), 3.82 (ddd, J=10.1, 6.2, 3.7 Hz, 1H), 2.13-2.01 (m, 2H), 1.85-1.69 (m, 2H), 1.69-1.59 (m, 1H), 1.54-1.22 (m, 5H). ¹³C NMR (75 MHz, Chloroform-d) δ 159.2, 149.3, 136.0, 123.0, 119.3, 43.0, 33.3, 26.0, 25.8. HRMS C₁₁H₁₅NS [M+H]⁺; calculated 194.0997, found: 194.0998. The spectral data are consistent with those reported in the literature.

### Example 18

### 2-(cyclohexylthio)pyrazine

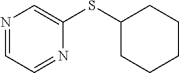 [SMILES: c1cnc(SC2CCCCC2)cn1]

Prepared by general procedure A for 2 h at 80° C.; isolated as a pale yellow liquid using pentane/ethyl acetate (50:1) as eluent (77.6 mg, 80%). ¹H NMR (300 MHz, Chloroform-d) δ 8.42 (d, J=1.6 Hz, 1H), 8.37 (dd, J=2.7, 1.6 Hz, 1H), 8.19 (d, J=2.7 Hz, 1H), 3.93-3.78 (m, 1H), 2.18-2.04 (m, 2H), 1.87-1.73 (m, 2H), 1.67 (ddt, J=9.6, 6.3, 2.4 Hz, 1H), 1.58-1.29 (m, 5H). ¹³C NMR (75 MHz, Chloroform-d) b 157.3, 144.2, 143.9, 139.2, 42.9, 33.1, 25.9, 25.7. HRMS C₁₀H₁₄N₂S [M+H]⁺; calculated 195.0950, found: 195.0950.

### Example 19

### Dodecyl(4-methoxyphenyl)sulfane

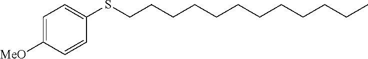 [SMILES: CCCCCCCCCCCCSc1ccc(OC)cc1]

Prepared by general procedure A; isolated as a pale yellow liquid using pentane/ethyl acetate (50:1) as eluent (135 mg, 88%). ¹H NMR (300 MHz, Chloroform-d) δ 7.35 (d, J=8.8 Hz, 2H), 6.86 (d, J=8.8 Hz, 2H), 3.82 (s, 3H), 2.84 (br, 2H), 1.72-1.50 (m, 2H), 1.43-1.28 (m, 18H), 0.98-0.82 (m, 3H). ¹³C NMR (126 MHz, CDCl₃) δ 158.7, 132.9, 114.8, 114.5, 55.3, 35.8, 31.9, 29.7, 29.63, 29.59, 29.5, 29.40, 29.35, 29.2, 28.7, 22.7, 14.1. HRMS C₁₉H₃₂OS [M]⁺; calculated 308.2168, found: 308.2167. The spectral data are consistent with those reported in the literature.

### Example 20

### ((3s,5s,7s)-adamantan-1-yl)(phenyl)sulfane

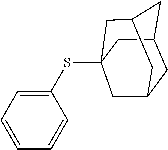 [SMILES: c1ccc(SC23CC4CC(CC(C4)C2)C3)cc1]

Prepared by general procedure A; isolated as a white solid using pentane as eluent (95 mg, 78%). ¹H NMR (500 MHz, Chloroform-d) δ 7.56-7.48 (m, 2H), 7.39-7.29 (m, 3H), 2.06-1.95 (m, 3H), 1.82 (d, J=2.9 Hz, 6H), 1.70-1.56 (m, 6H). ¹³C NMR (126 MHz, CDCl₃) δ 137.7, 130.5, 128.6, 128.3, 47.8, 43.6, 36.2, 30.0. HRMS C₁₆H₂OS [M]⁺; calculated 244.1280, found: 244.1280.

### Example 21

### Phenethyl(phenyl)sulfane

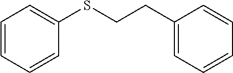 [SMILES: c1ccc(CCSc2ccccc2)cc1]

Prepared by general procedure A; isolated as a colorless liquid using pentane/ethyl acetate (100:1) as eluent (60.5 mg, 57%). ¹H NMR (500 MHz, Chloroform-d) δ 7.21-7.15 (m, 2H), 7.11 (td, J=7.7, 3.1 Hz, 4H), 7.07-6.96 (m, 4H), 3.04-2.93 (m, 2H), 2.80-2.69 (m, 2H). ¹³C NMR (126 MHz, CDCl₃) δ 140.2, 136.4, 129.2, 128.9, 128.6, 128.5, 126.5, 126.0, 35.7, 35.1. HRMS C₁₄H₁₄S [M]⁺; calculated 214.0816, found: 214.0816. The spectral data are consistent with those reported in the literature.

### Example 22

### Phenyl((1S,2S,5R)-2,6,6-trimethylbicyclo[3.1.1]heptan-2-yl)sulfane

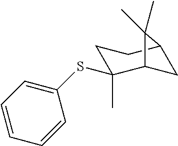 [SMILES: CC1(Sc2ccccc2)CCC2CC1C2(C)C]

Prepared by general procedure A; isolated as a colorless liquid using pentane/ethyl acetate (100:1) as eluent (114 mg, 93%). ¹H NMR (500 MHz, Chloroform-d) δ 7.41-7.32 (m, 2H), 7.32-7.27 (m, 2H), 7.24-7.13 (m, 1H), 3.19-2.79 (m, 2H), 2.46-2.24 (m, 2H), 2.14-1.82 (m, 5H), 1.74-1.53 (m, 1H), 1.22 (s, 3H), 1.06 (s, 3H), 0.91 (d, J=9.7 Hz, 1H). ¹³C NMR (126 MHz, CDCl₃) δ 137.3, 128.9, 128.8, 125.6, 45.6, 41.3, 40.8, 40.6, 38.7, 33.3, 28.0, 26.2, 23.3, 22.1. HRMS C₁₆H₂₂S [M]⁺; calculated 246.1436, found: 246.1437.

### Example 23

### Octyl(phenyl)sulfane

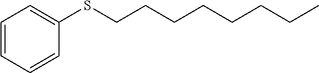

Prepared by general procedure A; isolated as a colorless liquid using pentane as eluent (102 mg, 92%). ¹H NMR (500 MHz, Chloroform-d) δ 7.15 (dd, J=8.6, 1.4 Hz, 2H), 7.09 (dd, J=8.6, 6.9 Hz, 2H), 7.01-6.94 (m, 1H), 2.82-2.69 (m, 2H), 1.63-1.40 (m, 2H), 1.33-1.20 (m, 2H), 1.16-1.03 (m, 8H), 0.71 (t, J=7.0 Hz, 3H). ¹³C NMR (126 MHz, CDCl₃) δ 137.1, 128.84, 128.81, 125.6, 33.6, 31.9, 29.23, 29.20, 29.19, 28.9, 22.7, 14.1. HRMS C₁₄H₂₂S [M]⁺; calculated 222.1442, found: 222.1441. The spectral data are consistent with those reported in the literature.

### Example 24

### Benzyl(phenyl)sulfane

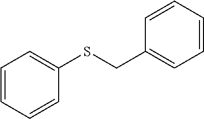 [SMILES: c1ccc(CSc2ccccc2)cc1]

Prepared by general procedure A for 1 h; isolated as a colorless liquid using pentane as eluent (76.6 mg, 77%). ¹H NMR (500 MHz, Chloroform-d) δ 7.37-7.26 (m, 9H), 7.25-7.19 (m, 1H), 4.16 (s, 2H). ¹³C NMR (126 MHz, CDCl₃) δ 137.5, 136.4, 129.8, 128.9, 128.8, 128.5, 127.2, 126.4, 39.1. HRMS C₁₃H₁₂S [M]⁺; calculated 200.0654, found: 200.0654. The spectral data are consistent with those reported in the literature (45).

### Example 25

### 1,3-bis(cyclohexylthio)benzene

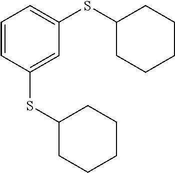 [SMILES: c1cc(SC2CCCCC2)cc(SC2CCCCC2)c1]

Prepared by general procedure A (0.2 mmol scale); isolated as a pale yellow liquid using pentane/ethyl acetate (100:1) as eluent (53.9 mg, 88%). ¹H NMR (500 MHz, Chloroform-d) δ 7.41 (s, 1H), 7.24-7.15 (m, 3H), 3.30-3.00 (m, 2H), 2.07-1.91 (m, 4H), 1.84-1.74 (m, 4H), 1.69-1.59 (m, 2H), 1.44-1.17 (m, 10H). ¹³C NMR (126 MHz, CDCl₃) δ 135.9, 134.2, 129.7, 128.9, 46.5, 33.3, 26.0, 25.8. HRMS C₁₈H₂₆S₂ [M]⁺; calculated 306.1476, found: 306.1478.

### Example 26

### ((3S,10R,13R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)(phenyl)sulfane

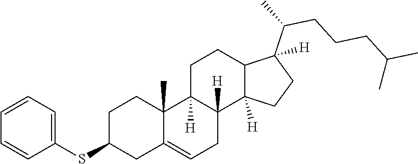

Prepared by general procedure A (0.2 mmol scale); isolated as a white solid using pentane as eluent (66.6 mg, 70%). ¹H NMR (500 MHz, Chloroform-d) δ 7.42 (dd, J=8.2, 1.3 Hz, 2H), 7.31 (dd, J=8.2, 6.8 Hz, 2H), 7.28-7.20 (m, 1H), 5.34 (dd, J=5.1, 1.7 Hz, 1H), 3.17-2.95 (m, 1H), 2.35 (d, J=8.2 Hz, 2H), 2.08-1.95 (m, 2H), 1.92 (dt, J=9.5, 3.7 Hz, 2H), 1.90-1.80 (m, 1H), 1.69-1.44 (m, 12H), 1.45-1.05 (m, 9H), 1.02 (s, 3H), 0.95 (d, J=6.5 Hz, 3H), 0.91 (d, J=2.2 Hz, 3H), 0.89 (d, J=2.2 Hz, 3H), 0.70 (s, 3H). ¹³C NMR (126 MHz, CDCl₃) δ 141.7, 134.9, 131.8, 128.8, 126.6, 121.2, 56.8, 56.2, 50.3, 47.4, 42.3, 39.8, 39.7, 39.5, 36.9, 36.2, 35.8, 31.9, 31.8, 29.5, 28.2, 28.0, 24.3, 23.9, 22.8, 22.6, 20.9, 19.4, 18.7, 11.9. [α]_{D}²⁵=−37.7 (c=0.52, CH₂Cl₂). HRMS C₃₃H₅₀S [M]⁺; calculated 478.3633, found: 478.3634.

### Example 27

### Cyclohexyl(phenyl)sulfane

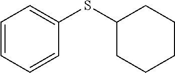 [SMILES: c1ccc(SC2CCCCC2)cc1]

Prepared by general procedure A; isolated as a colorless liquid using pentane as eluent (86.2 mg, 90%). ¹H NMR (500 MHz, Chloroform-d) δ 7.41-7.38 (m, 2H), 7.30-7.26 (m, 2H), 7.23-7.18 (m, 1H), 3.17-3.01 (m, 1H), 2.02-1.95 (m, 2H), 1.82-1.73 (m, 2H), 1.66-1.58 (m, 1H), 1.44-1.24 (m, 5H). ¹³C NMR (125 MHz, CDCl₃) δ 135.2, 131.9, 128.7, 126.6, 46.6, 33.4, 26.1, 25.8. HRMS C₁₂H₁₆S [M]⁺; calculated 192.0967, found: 192.0969. The spectral data are consistent with those reported in the literature.

### Example 28

### Cyclohexyl(styryl)sulfane

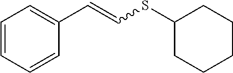 [SMILES: C(=Cc1ccccc1)SC1CCCCC1]

Prepared from the corresponding starting material (E/Z 5:1) by general procedure A at 160° C.; isolated as a pale yellow liquid using pentane/ethyl acetate (100:1) as eluent (89 mg, 82%, E/Z=5:1). E isomer: ¹H NMR (500 MHz, Chloroform-d) b 7.35-7.30 (m, 4H), 7.25-7.19 (m, 1H), 6.80 (d, J=15.6 Hz, 1H), 6.61 (d, J=15.6 Hz, 1H), 3.05-3.01 (m, 1H), 2.20-2.03 (m, 2H), 1.92-1.78 (m, 2H), 1.73-1.64 (m, 1H), 1.55-1.07 (m, 6H). ¹³C NMR (126 MHz, CDCl₃) δ 137.2, 128.6, 128.6, 126.9, 125.6, 124.1, 45.3, 33.6, 26.0, 25.7. Z isomer: ¹H NMR (500 MHz, Chloroform-d) b 7.58-7.49 (m, 2H), 7.38 (t, J=7.8 Hz, 2H), 7.24 (s, 1H), 6.47 (d, J=11.0 Hz, 1H), 6.37 (d, J=11.0 Hz, 0H), 2.97-2.88 (m, 1H), 2.09-2.07 (m, 2H), 1.85-1.82 (m, 2H), 1.69-1.66 (m, 1H), 1.58-1.28 (m, 6H). ¹³C NMR (126 MHz, CDCl₃, characteristic peak) 137.2, 128.2, 126.5, 125.9, 125.0, 47.8, 33.7, 25.6. HRMS C₁₄H₁₈S [M]⁺; calculated 218.1129, found: 218.1129. The spectral data are consistent with those reported in the literature.

General Procedure for the Catalytic Aryl Transfer with Thiophenols

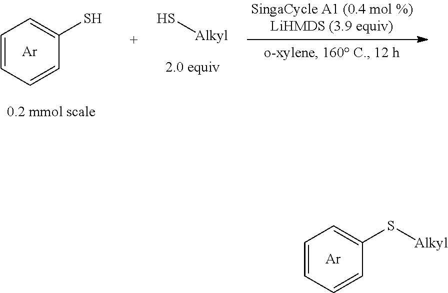

In the glovebox, aryl thiol (0.2 mmol), alkyl thiol (2.0 equiv, 0.4 mmol), LiHMDS (3.9 equiv, 135 mg), and SingaCycle A1 (0.4 mol %, 0.2 ml, 0.005 M in o-xylene) were added into the oven-dried 8 ml vial with a magnetic stirring bar, followed by addition of o-xylene (1.8 ml). The vial was sealed and removed out of the glovebox and heated to 160° C. After 12 h, the vial was cooled to room temperature. The reaction was diluted with ethyl acetate and washed by saturated NaOH solution. The aqueous phase was extracted with ethyl acetate 3 times. The collected organic phases were dried over anhydrous Na₂SO₄. The solvent was removed under reduced pressure and the residue was purified by flash column chromatography to give the desired product.

### Example 29

### (4-methoxyphenyl)(octyl)sulfane

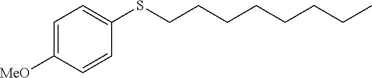 [SMILES: CCCCCCCCSc1ccc(OC)cc1]

Prepared by general procedure B; isolated as a pale yellow liquid using pentane/ethyl acetate (50:1) as eluent (38 mg, 76%). ¹H NMR (300 MHz, Chloroform-d) δ 7.34 (d, J=8.9 Hz, 2H), 6.84 (d, J=8.8 Hz, 2H), 3.79 (s, 3H), 2.99-2.71 (m, 2H), 1.65-1.50 (m, 2H), 1.45-1.17 (m, 10H), 0.97-0.75 (m, 3H). ¹³C NMR (75 MHz, CDCl₃) δ 158.7, 132.8, 127.0, 114.4, 55.3, 35.8, 31.8, 29.3, 29.1, 29.0, 28.7, 22.6, 14.1. HRMS C₁₅H₂₄SO [M]⁺; calculated 252.1542, found: 252.1544. The spectral data are consistent with those reported in the literature.

### Example 30

### Cyclohexyl(p-tolyl)sulfane

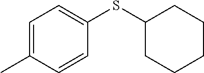

Prepared by general procedure B; isolated as a colorless liquid using pentane/ethyl acetate (100:1) as eluent (30.5 mg, 75%). See above for experimental data.

### Example 31

### Cyclopentyl(o-tolyl)sulfane

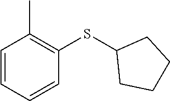

Prepared by general procedure B; isolated as a colorless liquid using pentane/ethyl acetate (100:1) as eluent (29.3 mg, 77%). ¹H NMR (500 MHz, Chloroform-d) δ 7.39-7.29 (m, 1H), 7.15 (t, J=7.7 Hz, 2H), 7.11-7.06 (m, 1H), 3.71-3.49 (m, 1H), 2.37 (s, 3H), 2.17-2.03 (m, 2H), 1.84-1.75 (m, 2H), 1.72-1.56 (m, 4H). ¹³C NMR (126 MHz, CDCl₃) δ 137.5, 136.8, 130.0, 128.8, 126.2, 125.4, 44.9, 33.6, 24.9, 20.5. HRMS C₁₂H₁₆S [M]⁺; calculated 192.0973, found: 192.0970. The spectral data are consistent with those reported in the literature.

### Example 32

### Cyclohexyl(phenyl)sulfane

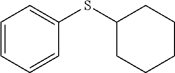 [SMILES: c1ccc(SC2CCCCC2)cc1]

Prepared by general procedure B; isolated as a colorless liquid using pentane as eluent (32.3 mg, 85%). See above for experimental data.

### Example 33

### (4-(tert-butyl)phenyl)(2-methylbutyl)sulfane

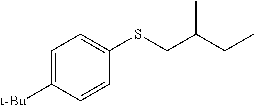 [SMILES: CCC(C)CSc1ccc(C(C)(C)C)cc1]

Prepared by general procedure B; isolated as a colorless liquid using pentane/ethyl acetate (100:1) as eluent (42.6 mg, 91%). ¹H NMR (500 MHz, Chloroform-d) δ 7.44-7.27 (m, 4H), 2.97 (dd, J=12.5, 5.9 Hz, 1H), 2.77 (dd, J=12.5, 7.5 Hz, 1H), 1.87-1.66 (m, 1H), 1.63-1.51 (m, 1H), 1.34 (s, 9H), 1.32-1.24 (m, 1H), 1.06 (d, J=6.7 Hz, 3H), 0.94 (t, J=7.4 Hz, 3H). ¹³C NMR (126 MHz, CDCl₃) δ 148.8, 133.9, 128.9, 125.8, 41.2, 34.6, 34.4, 31.3, 28.8, 18.9, 11.3. HRMS C₁₅H₂₄S [M]⁺; calculated 236.1599, found: 236.1599.

### Example 34

### 4-(cyclohexylthio)pyridine

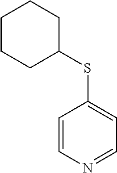 [SMILES: c1cc(SC2CCCCC2)ccn1]

Prepared by general procedure B; isolated as a colorless liquid using pentane/ethyl acetate (20:1) as eluent (18 mg, 47%). ¹H NMR (500 MHz, Chloroform-d) δ 8.37 (d, J=6.3 Hz, 2H), 7.11 (d, J=6.3 Hz, 2H), 3.48-3.25 (m, 1H), 2.10-1.95 (m, 2H), 1.86-1.74 (m, 2H), 1.68-1.64 (m, 1H), 1.54-1.35 (m, 5H). ¹³C NMR (126 MHz, CDCl₃) δ 149.1, 148.8, 121.7, 43.4, 32.9, 25.8, 25.6. HRMS C₁₁H₁₅NS [M+H]⁺; calculated 194.0997, found: 194.0997. The spectral data are consistent with those reported in the literature.

### Example 35

### (3,5-dimethylphenyl)(2-methylbutyl)sulfane

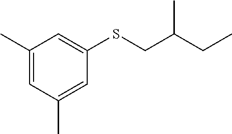

Prepared by general procedure B; isolated as a colorless liquid using pentane/ethyl acetate (100:1) as eluent (37.6 mg, 91%). ¹H NMR (500 MHz, Chloroform-d) δ 6.98 (s, 2H), 6.82 (s, 1H), 2.97 (dd, J=12.4, 5.8 Hz, 1H), 2.77 (dd, J=12.4, 7.5 Hz, 1H), 2.32 (s, 6H), 1.78-1.65 (m, 1H), 1.63-1.54 (m, 1H), 1.31 (dt, J=13.5, 7.5 Hz, 1H), 1.06 (d, J=6.6 Hz, 3H), 0.95 (t, J=7.5 Hz, 3H). ¹³C NMR (126 MHz, CDCl₃) δ 138.4, 137.1, 127.4, 126.4, 40.6, 34.6, 28.8, 21.3, 19.0, 11.3. HRMS C₁₃H₂OS [M]⁺; calculated 208.1286, found: 208.1284. The spectral data are consistent with those reported in the literature.

### Example 36

### (4-methoxyphenyl)(2-methylbutyl)sulfane

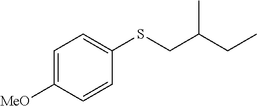

Prepared by general procedure B; isolated as a pale yellow liquid using pentane/ethyl acetate (40:1) as eluent (33.9 mg, 81%). ¹H NMR (300 MHz, Chloroform-d) δ 7.41-7.27 (m, 2H), 6.91-6.78 (m, 2H), 3.79 (s, 3H), 2.85 (dd, J=12.6, 5.7 Hz, 1H), 2.66 (dd, J=12.6, 7.4 Hz, 1H), 1.68-1.45 (m, 2H), 1.35-1.17 (m, 1H), 0.99 (d, J=6.6 Hz, 3H), 0.87 (t, J=7.4 Hz, 3H). ¹³C NMR (75 MHz, CDCl₃) δ 158.6, 132.6, 127.6, 114.5, 55.3, 43.0, 34.5, 28.6, 18.8, 11.2. HRMS C₁₂H₁₈SO [M]⁺; calculated 210.1078, found: 210.1079.

### Example 37

### 2-((2-methylbutyl)thio)-5-phenylbenzo[d]thiazole

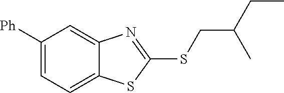 [SMILES: CCC(C)CSc1nc2cc(-c3ccccc3)ccc2s1]

Prepared by general procedure B; isolated as a pink solid using pentane/ethyl acetate (50:1) as eluent (23 mg, 37%). ¹H NMR (300 MHz, Chloroform-d) δ 8.08 (s, 1H), 7.80 (d, J=8.3 Hz, 1H), 7.66 (d, J=7.5 Hz, 2H), 7.55-7.37 (m, 4H), 3.44 (dd, J=12.7, 5.9 Hz, 1H), 3.24 (dd, J=12.7, 7.4 Hz, 1H), 1.97-1.76 (m, 1H), 1.71-1.52 (m, 1H), 1.39-1.30 (m, 1H), 1.08 (d, J=6.7 Hz, 3H), 0.97 (t, J=7.4 Hz, 3H). ¹³C NMR (126 MHz, CDCl₃) δ 168.5, 154.0, 140.8, 139.6, 134.1, 128.9, 127.4, 127.3, 123.5, 121.0, 119.8, 40.4, 34.9, 28.7, 18.9, 11.3. HRMS C₁₃H₁₉S₂N [M]⁺; calculated 314.1032, found: 314.1033.

### Example 38

### Cyclohexyl(naphthalen-1-yl)sulfane

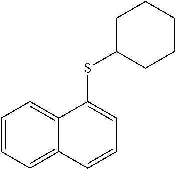 [SMILES: c1ccc2c(SC3CCCCC3)cccc2c1]

Prepared by general procedure B; isolated as a colorless liquid using pentane/ethyl acetate (100:1) as eluent (33.5 mg, 70%). ¹H NMR (300 MHz, Chloroform-d) δ 8.63-8.49 (m, 1H), 7.89-7.75 (m, 2H), 7.71 (dd, J=7.2, 1.2 Hz, 1H), 7.55 (ddd, J=10.5, 7.9, 1.4 Hz, 2H), 7.42 (dd, J=8.2, 7.2 Hz, 1H), 3.31-3.04 (m, 1H), 2.06-1.92 (m, 2H), 1.82-1.73 (m, 2H), 1.65-1.57 (m, 1H), 1.52-1.12 (m, 5H). ¹³C NMR (75 MHz, CDCl₃) δ 134.4, 134.0, 132.3, 131.8, 128.4, 128.1, 126.3, 126.0, 125.9, 125.4, 47.2, 33.5, 26.0, 25.8. HRMS C₁₆H₁₈S [M]⁺; calculated 242.1124, found: 242.1123.

### Example 39

### Cyclohexyl(naphthalen-2-yl)sulfane

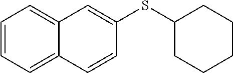 [SMILES: c1ccc2cc(SC3CCCCC3)ccc2c1]

Prepared by general procedure B; isolated as a colorless liquid using pentane/ethyl acetate (100:1) as eluent (40 mg, 83%). ¹H NMR (300 MHz, Chloroform-d) δ 7.87 (d, J=1.8 Hz, 1H), 7.79 (ddd, J=12.7, 7.9, 2.4 Hz, 3H), 7.56-7.41 (m, 3H), 3.37-3.17 (m, 1H), 2.19-1.99 (m, 2H), 1.85-1.73 (m, 2H), 1.71-1.59 (m, 1H), 1.52-1.20 (m, 5H). ¹³C NMR (75 MHz, CDCl₃) δ 133.6, 132.6, 132.0, 130.1, 129.6, 128.2, 127.6, 127.2, 126.3, 125.8, 46.5, 33.3, 26.0, 25.8. HRMS C₁₆H₁₈S [M]⁺; calculated 242.1124, found: 242.1123.

### Example 40

### ((3s,5s,7s)-adamantan-1-yl)(phenyl)sulfane

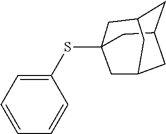 [SMILES: c1ccc(SC23CC4CC(CC(C4)C2)C3)cc1]

Prepared by general procedure B; isolated as a white solid using pentane as eluent (34.1 mg, 70%). See above for experimental data.

### Example 41

### Phenyl((1S,2S,5R)-2,6,6-trimethylbicyclo[3.1.1]heptan-2-yl)sulfane

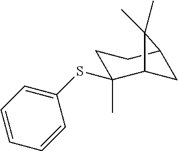 [SMILES: CC1(Sc2ccccc2)CCC2CC1C2(C)C]

Prepared by general procedure B; isolated as a colorless liquid using pentane/ethyl acetate (100:1) as eluent (45 mg, 92%). See above for experimental data.

### Example 42

### N,N-dimethyl-2-(phenylthio)ethan-1-amine

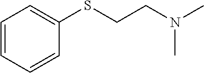

Prepared by general procedure B; isolated as a pale yellow liquid using acetone as eluent (21.6 mg, 60%). ¹H NMR (300 MHz, Chloroform-d) δ 7.41-7.34 (m, 2H), 7.33-7.26 (m, 2H), 7.23-7.15 (m, 1H), 3.20-2.93 (m, 2H), 2.83-2.52 (m, 2H), 2.30 (s, 6H). ¹³C NMR (75 MHz, CDCl₃) δ 136.4, 128.9, 128.8, 125.8, 58.5, 45.3, 31.4. HRMS C₁₀H₁₅SN [M+H]⁺; calculated 182.0998, found: 182.0999.

### Example 43

### (2-((3r,5r,7r)-adamantan-1-yl)ethyl)(phenyl)sulfane

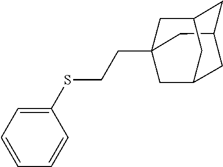 [SMILES: c1ccc(SCCC23CC4CC(CC(C4)C2)C3)cc1]

Prepared by general procedure B; isolated as a colorless liquid using pentane/ethyl acetate (100:1) as eluent (39.3 mg, 73%). ¹H NMR (500 MHz, Chloroform-d) δ 7.39-7.27 (m, 4H), 7.24-7.14 (m, 1H), 3.05-2.76 (m, 2H), 1.99 (q, J=3.0 Hz, 3H), 1.82-1.71 (m, 3H), 1.70-1.63 (m, 3H), 1.55 (d, J=3.0 Hz, 6H), 1.52-1.37 (m, 2H). ¹³C NMR (126 MHz, CDCl₃) δ 137.2, 128.8, 128.4, 125.5, 43.6, 42.2, 37.1, 32.8, 28.6, 27.5. HRMS C₁₈H₂₄S [M]⁺; calculated 272.1599, found: 272.1597.

### Example 44

### Phenethyl(phenyl)sulfane

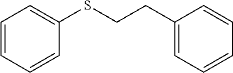 [SMILES: c1ccc(CCSc2ccccc2)cc1]

Prepared by general procedure B; isolated as a colorless liquid using pentane/ethyl acetate (100:1) as eluent (30.2 mg, 71%). See above for experimental data.

### Example 45

### Diphenylsulfane

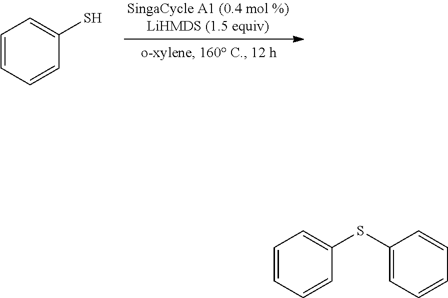 [SMILES: Sc1ccccc1 ; c1ccc(Sc2ccccc2)cc1]

In the glovebox, phenyl thiol (0.2 mmol), LiHMDS (1.5 equiv, 51.6 mg), and SingaCycle A1 (0.4 mol %) were added into an oven-dried 8 ml vial with a magnetic stirring bar, followed by addition of o-xylene (2 ml). The vial was sealed and removed out of the glovebox and heated to 160° C. After 12 h, the vial was cooled to room temperature. The reaction was diluted with ethyl acetate and washed by saturated NaOH solution. The aqueous phase was extracted with ethyl acetate 3 times. The collected organic phases were dried over anhydrous Na₂SO₄. The solvent was removed under reduced pressure and the residue was purified by flash column chromatography (pentane) to give the title product (14.1 mg, 76%). ¹H NMR (500 MHz, Chloroform-d) δ 7.41-7.36 (m, 4H), 7.36-7.31 (m, 4H), 7.30-7.25 (m, 2H). ¹³C NMR (126 MHz, CDCl₃) δ 135.8, 131.1, 129.2, 127.0. HRMS C₁₂H₁₀S [M]⁺; calculated 186.0497, found: 186.0498. The spectral data are consistent with those reported in the literature.

### Example 46

### Diphenylselane

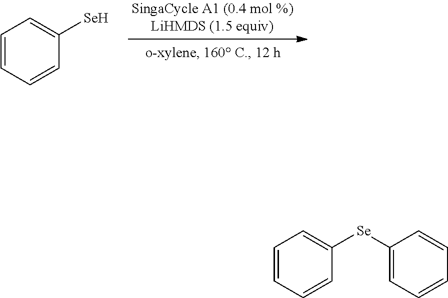

In the glovebox, phenyl selenol (0.2 mmol), LiHMDS (1.5 equiv, 51.6 mg), and SingaCycle A1 (0.4 mol %) were added into an oven-dried 8 ml vial with a magnetic stirring bar, followed by addition of o-xylene (2 ml). The vial was sealed and removed out of the glovebox and heated to 160° C. After 12 h, the vial was cooled to room temperature. The reaction was diluted with ethyl acetate and washed by saturated NaOH solution. The aqueous phase was extracted with ethyl acetate 3 times. The collected organic phases were dried over anhydrous Na₂SO₄. The solvent was removed under reduced pressure and the residue was purified by flash column chromatography (pentane) to give the title product (22.3 mg, 96%). ¹H NMR (500 MHz, Chloroform-d) δ 7.53 (dq, J=7.7, 3.5 Hz, 4H), 7.31 (dq, J=5.7, 3.0 Hz, 6H). ¹³C NMR (126 MHz, Chloroform-d) δ 133.1 (d, J=3.0 Hz), 131.2 (d, J=4.3 Hz), 129.4 (d, J=3.8 Hz), 127.4 (d, J=2.7 Hz). HRMS C₁₂H₁₀Se [M]⁺; calculated 233.9947, found: 233.9948. The spectral data are consistent with those reported in the literature.

### Procedure for Late-Stage Derivatization

### Example 47

### 2-(cyclohexylthio)-10-(2-(1-methylpiperidin-2-yl)ethyl)-10H-phenothiazine

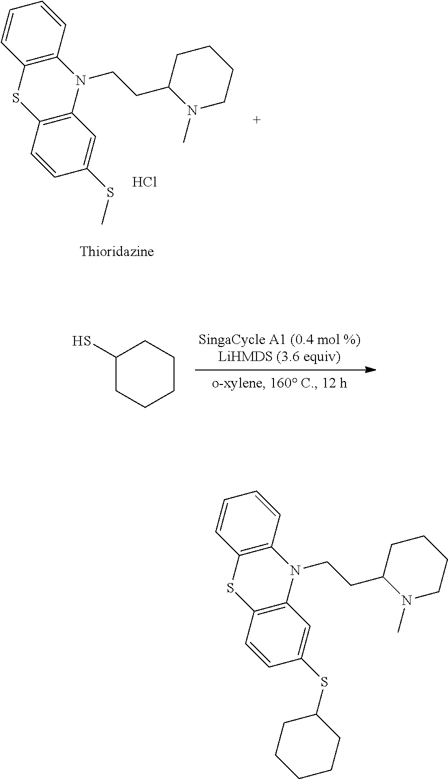 [SMILES: CSc1ccc2c(c1)N(CCC1CCCCN1C)c1ccccc1S2 ; Cl ; SC1CCCCC1 ; CN1CCCCC1CCN1c2ccccc2Sc2ccc(SC3CCCCC3)cc21]

In the glovebox, thioridazine (0.2 mmol), cyclohexyl thiol (2.0 equiv, 0.4 mmol), LiHMDS (3.6 equiv), and SingaCycle A1 (0.4 mol %) were added into an oven-dried 8 ml vial with a magnetic stirring bar, followed by addition of o-xylene (1.0 ml). The vial was sealed and removed out of the glovebox and heated to 160° C. After 12 h, the vial was cooled to room temperature. The reaction was diluted with ethyl acetate and washed with NaOH solution. The aqueous phase was extracted with ethyl acetate 3 times. The collected organic phases were dried over anhydrous Na₂SO₄. The solvent was removed under reduced pressure and the residue was purified by flash column chromatography (0-5% MeOH in DCM) to give the title product. Isolated as syrup like liquid (54.3 mg, 62%). ¹H NMR (500 MHz, Chloroform-d) δ 7.21-7.11 (m, 2H), 7.05 (d, J=7.9 Hz, 1H), 6.98 (dd, J=7.9, 1.7 Hz, 1H), 6.93 (dd, J=11.5, 1.7 Hz, 2H), 6.89 (dd, J=7.9, 1.1 Hz, 1H), 4.09-3.92 (m, 1H), 3.86 (dt, J=14.2, 7.4 Hz, 1H), 3.14-2.99 (m, 1H), 2.97-2.86 (m, 1H), 2.27-2.23 (m, 6H), 2.04-1.88 (m, 3H), 1.83-1.55 (m, 7H), 1.49-1.12 (m, 7H). ¹³C NMR (126 MHz, CDCl₃) δ 145.4, 144.9, 134.1, 127.6, 127.5, 127.4, 126.5, 125.4, 124.5, 122.8, 119.7, 115.9, 62.3, 56.7, 47.2, 43.8, 42.4, 33.3, 30.2, 29.3, 26.0, 25.7, 24.9, 23.6. HRMS C₂₆H₃₄S₂N₂ [M+H]⁺; calculated 439.2236, found: 439.2239.

### Example 48

### 10-(2-(1-methylpiperidin-2-yl)ethyl)-2-(octylthio)-10H-phenothiazine

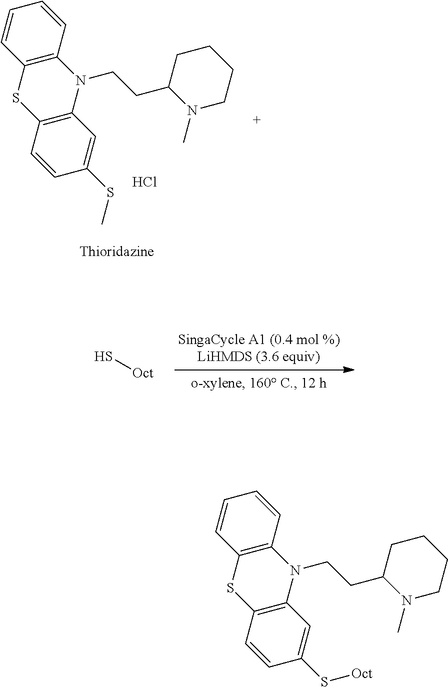

In the glovebox, thioridazine (0.2 mmol), 1-octanethiol (2.0 equiv, 0.4 mmol), LiHMDS (3.6 equiv), and SingaCycle A1 (0.4 mol %) were added into an oven-dried 8 ml vial with a magnetic stirring bar, followed by addition of o-xylene (1.0 ml). Then the vial was sealed and removed out of the glovebox and heated to 160° C. After 12 h, the vial was cooled to room temperature. The reaction was diluted with ethyl acetate and washed with NaOH solution. The aqueous phase was extracted with ethyl acetate 3 times. The collected organic phases were dried over anhydrous Na₂SO₄. The solvent was removed under reduced pressure and the residue was purified by flash column chromatography (0-5% MeOH in DCM) to give the title product. Isolated as orange oil (46 mg, 49%). ¹H NMR (500 MHz, Chloroform-d) δ 7.20-7.09 (m, 2H), 7.04 (d, J=7.9 Hz, 1H), 6.97-6.81 (m, 4H), 3.96 (ddd, J=13.8, 8.5, 5.3 Hz, 1H), 3.84 (ddd, J=13.8, 8.7, 6.1 Hz, 1H), 2.93-2.81 (m, 3H), 2.24 (s, 3H), 2.15 (qt, J=9.8, 7.2, 3.3 Hz, 3H), 1.89 (dtd, J=13.8, 8.0, 4.8 Hz, 1H), 1.77-1.69 (m, 2H), 1.66-1.57 (m, 4H), 1.55-1.43 (m, 1H), 1.43-1.35 (m, 2H), 1.35-1.19 (m, 9H), 0.87 (t, J=6.9 Hz, 3H). ¹³C NMR (126 MHz, CDCl₃) δ 145.8, 145.1, 136.2, 127.7, 127.6, 127.4, 125.5, 123.4, 123.2, 122.8, 117.0, 115.9, 62.4, 57.0, 44.0, 43.0, 34.4, 31.9, 30.7, 29.8, 29.3, 29.3, 29.3, 29.0, 25.5, 24.1, 22.8, 14.2. HRMS C₂₈H₄₀S₂N₂ [M+H]⁺; calculated 469.2705, found: 469.2709.

### Example 49

### 2-(benzylthio)-10-(2-(1-methylpiperidin-2-yl)ethyl)-10H-phenothiazine

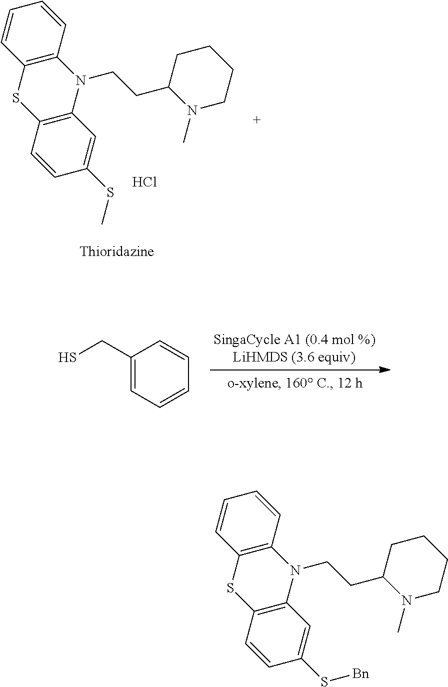 [SMILES: CSc1ccc2c(c1)N(CCC1CCCCN1C)c1ccccc1S2 ; Cl ; SCc1ccccc1 ; CN1CCCCC1CCN1c2ccccc2Sc2ccc(SCc3ccccc3)cc21]

In the glovebox, thioridazine (0.2 mmol), benzyl thiol (2.0 equiv, 0.4 mmol), LiHMDS (3.6 equiv), and SingaCycle A1 (0.4 mol %) were added into an oven-dried 8 ml vial with a magnetic stirring bar, followed by addition of o-xylene (1.0 ml). The vial was sealed and removed out of the glovebox and heated to 160° C. After 12 h, the vial was cooled to room temperature. The reaction was diluted with ethyl acetate and washed with NaOH solution. The aqueous phase was extracted with ethyl acetate 3 times. The collected organic phases were dried over anhydrous Na₂SO₄. The solvent was removed under reduced pressure and the residue was purified by flash column chromatography (0-5% MeOH in DCM) to give the title product. Isolated as orange oil (49.7 mg, 56%). ¹H NMR (500 MHz, Chloroform-d) δ 7.22-7.12 (m, 5H), 7.10-7.01 (m, 2H), 6.93 (d, J=8.0 Hz, 1H), 6.87-6.77 (m, 2H), 6.76 (dd, J=8.0, 1.2 Hz, 1H), 6.65 (d, J=1.8 Hz, 1H), 3.98 (s, 2H), 3.79 (ddd, J=13.1, 7.7, 5.0 Hz, 1H), 3.64 (dt, J=14.3, 7.7 Hz, 1H), 2.89 (d, J=11.8 Hz, 1H), 2.22-2.06 (m, 6H), 1.86-1.47 (m, 6H), 1.32-1.11 (m, 1H). ¹³C NMR (126 MHz, CDCl₃) δ 145.5, 144.6, 137.5, 135.4, 128.81, 128.80, 128.6, 127.6, 127.5, 127.3, 125.6, 124.4, 124.1, 122.9, 117.7, 116.0, 62.4, 56.5, 43.6, 42.0, 39.4, 29.7, 28.7, 24.4, 23.3. HRMS C₂₇H₃₀S₂N₂ [M+H]⁺; calculated 447.1923, found: 447.1925.

### Example 50

### 2-(sec-butylthio)-10-(2-(1-methylpiperidin-2-yl)ethyl)-10H-phenothiazine

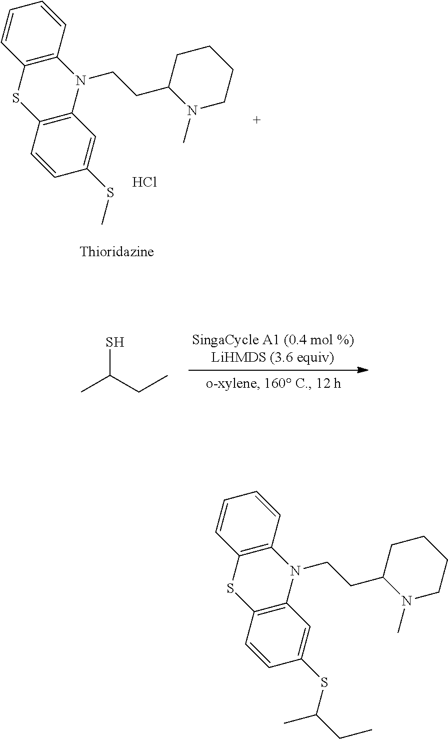

In the glovebox, thioridazine (0.2 mmol), sec-butyl thiol (2.0 equiv, 0.4 mmol), LiHMDS (3.6 equiv), and SingaCycle A1 (0.4 mol %) were added into an oven-dried 8 ml vial with a magnetic stirring bar, followed by addition of o-xylene (1.0 ml). The vial was sealed and removed out of the glovebox and heated to 160° C. After 12 h, the vial was cooled to room temperature. The reaction was diluted with ethyl acetate and washed with NaOH solution. The aqueous phase was extracted with ethyl acetate 3 times. The collected organic phases were dried over anhydrous Na₂SO₄. The solvent was removed under reduced pressure and the residue was purified by flash column chromatography (0-5% MeOH in DCM) to give the title product. Isolated as orange oil (58.3 mg, 71%). ¹H NMR (500 MHz, Chloroform-d) δ 7.19-7.10 (m, 2H), 7.04 (d, J=7.9 Hz, 1H), 6.96 (dd, J=7.9, 1.7 Hz, 1H), 6.95-6.88 (m, 2H), 6.91-6.85 (m, 1H), 3.96 (ddd, J=13.8, 8.5, 5.3 Hz, 1H), 3.85 (ddd, J=13.8, 8.5, 6.3 Hz, 1H), 3.10 (q, J=6.3 Hz, 1H), 2.88 (d, J=11.3 Hz, 1H), 2.24 (s, 3H), 2.21-2.10 (m, 3H), 1.89 (d, J=8.5 Hz, 1H), 1.78-1.69 (m, 2H), 1.67-1.58 (m, 3H), 1.58-1.47 (m, 2H), 1.36-1.26 (m, 1H), 1.25 (d, J=6.3 Hz, 3H), 0.99 (t, J=7.4 Hz, 3H). ¹³C NMR (126 MHz, CDCl₃) δ 145.4, 144.9, 134.4, 127.5, 127.43, 127.35, 126.3, 125.2, 124.3, 122.7, 119.6, 115.8, 62.2, 56.8, 45.5 (d, J=1.8 Hz), 43.9, 42.7, 30.5, 29.6, 29.5, 25.3, 23.9, 20.5, 11.5. HRMS C₂₄H₃₂S₂N₂ [M+H]⁺; calculated 413.2079, found: 413.2083.

### Example 51

### 2-(((1s,3s)-adamantan-1-yl)thio)-10-(2-(1-methylpiperidin-2-yl)ethyl)-10H-phenothiazine

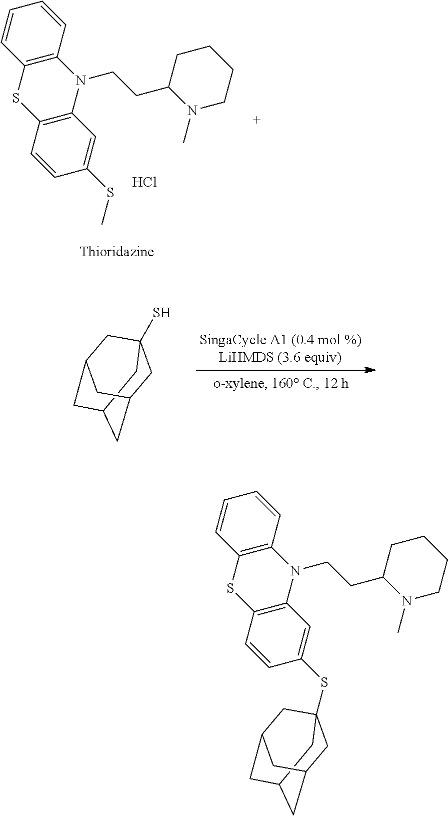 [SMILES: CSc1ccc2c(c1)N(CCC1CCCCN1C)c1ccccc1S2 ; Cl ; SC12CC3CC(CC(C3)C1)C2 ; CN1CCCCC1CCN1c2ccccc2Sc2ccc(SC34CC5CC(CC(C5)C3)C4)cc21]

In the glovebox, thioridazine (0.2 mmol), 1-adamentanethiol (2.0 equiv, 0.4 mmol), LiHMDS (3.6 equiv), and SingaCycle A1 (0.4 mol %) were added into an oven-dried 8 ml vial with a magnetic stirring bar, followed by addition of o-xylene (1.0 ml). The vial was sealed and removed out of the glovebox and heated to 160° C. After 12 h, the vial was cooled to room temperature. The reaction was diluted with ethyl acetate and washed with NaOH solution. The aqueous phase was extracted with ethyl acetate 3 times. The collected organic phases were dried over anhydrous Na₂SO₄. The solvent was removed under reduced pressure and the residue was purified by flash column chromatography (0-5% MeOH in DCM) to give the title product. Isolated as syrup like liquid (59.4 mg, 61%). ¹H NMR (500 MHz, Chloroform-d) δ 7.20-7.11 (m, 2H), 7.08-7.03 (m, 2H), 6.98 (d, J=1.3 Hz, 1H), 6.97-6.87 (m, 2H), 3.98 (ddd, J=13.6, 8.2, 5.2 Hz, 1H), 3.87 (ddd, J=14.2, 8.2, 6.6 Hz, 1H), 3.00-2.82 (m, 1H), 2.52-2.18 (m, 6H), 2.07-1.98 (m, 3H), 1.97-1.86 (m, 1H), 1.80 (d, J=2.8 Hz, 6H), 1.74-1.71 (m, 2H), 1.69-1.52 (m, 8H), 1.54-1.47 (m, 1H), 1.37-1.17 (m, 1H). ¹³C NMR (126 MHz, CDCl₃) δ 144.9, 131.7, 129.5, 127.6, 127.5, 127.4, 126.9, 126.6, 125.0, 124.4, 122.8, 115.8, 62.2, 56.7, 48.2, 43.9, 43.7, 42.5, 36.1, 30.2, 30.0, 29.4, 25.1, 23.7. HRMS C₃₀H₃₈S₂N₂ [M+H]⁺; calculated 491.2549, found: 491.2552.

### Example 52—Procedure for Depolymerization

### 1,4-bis(cyclopentylthio)benzene

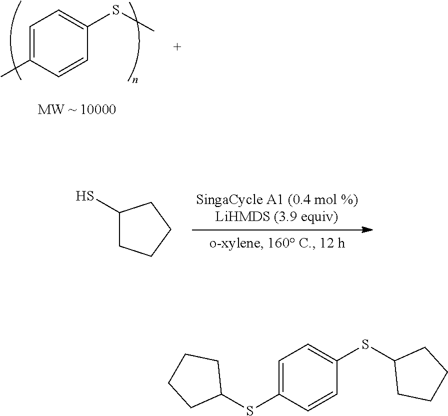

In the glovebox, poly(1,4-phenylene sulfide (MW-10000, 0.5 equiv, 0.1 mmol), 1-octanethiol (2.0 equiv, 0.4 mmol), LiHMDS (3.9 equiv), and SingaCycle A1 (0.4 mol %) were added into an oven-dried 8 ml vial with a magnetic stirring bar, followed by addition of o-xylene (2.0 ml). The vial was sealed and removed out of the glovebox and heated to 160° C. After 12 h, the vial was cooled to room temperature. The reaction was diluted with ethyl acetate and washed with NaOH solution. The aqueous phase was extracted with ethyl acetate 3 times. The collected organic phases were dried over anhydrous Na₂SO₄. The solvent was removed under reduced pressure and the residue was purified by flash column chromatography (pentane/ethyl acetate 40:1) to give the title product. Isolated as a pale yellow liquid (24.5 mg, 89%). ¹H NMR (500 MHz, Chloroform-d) δ 7.19 (d, J=1.3 Hz, 4H), 3.54-3.43 (m, 2H), 2.03-1.92 (m, 4H), 1.79-1.66 (m, 4H), 1.61-1.48 (m, 8H). ¹³C NMR (126 MHz, CDCl₃) δ 134.9, 130.5, 46.2, 33.5, 24.8. HRMS C₁₆H₂₂S2 [M]⁺; calculated 278.1163, found: 278.1166.

### Preparation Examples for the Nickel Catalyzed C/S Bond Metathesis by Arylation

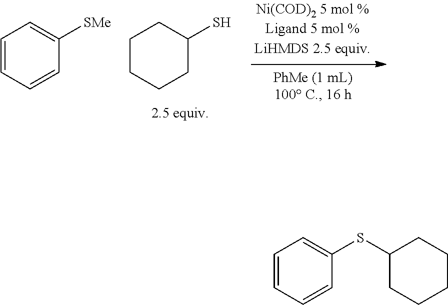

General Procedure

In a glovebox, an oven-dried 8 mL vial was charged with Bis-(dicyclohexylphosphino)-ethan (10.57 mg, 0.025 mmol, 5 mol %), Bis-(1,5-cyclooctadien)-nickel(0) (6.88 mg, 0.025 mmol, 5 mol %), Lithium bis(trimethylsilyl)amide (209.16 mg, 1.25 mmol, 2.5 equiv.), Methylthio arene (0.5 mmol, 1 equiv.), Alkylthiol (1.25 mmol, 2.5 equiv.) and toluene (1.25 mL, 0.4 mol/L). The reaction mixture was stirred at 100° C. for 12 h. The reaction mixture was allowed to cool to room temperature and was diluted with EtOAc and an aqueous solution of NaOH (1 mol/L). The layers were separated and the aqueous layer was extracted three times with EtOAc. The combined organic layers were dried over anhydrous MgSO₄ and concentrated to dryness. The residue was purified by FC (SiO₂, n-pentane to n-pentane:MTBE) to afford the title compound.

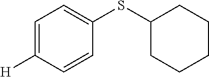 [SMILES: c1ccc(SC2CCCCC2)cc1]

### Example 53

### Cyclohexyl(phenyl)sulfane (1a)

Following the general procedure using thioanisole (62.1 mg, 0.5 mmol) and cyclohexanethiol (145.3 mg, 1.25 mmol). Purification by FC (SiO₂, n-pentane to n-pentane:MTBE 100:1) to afford the title compound (85.6 mg, 89%) as a colorless liquid. ¹H NMR (501 MHz, Chloroform-d) δ 7.42-7.37 (m, 2H), 7.31-7.26 (m, 2H), 7.24-7.19 (m, 1H), 3.11 (tt, J=10.5, 3.7 Hz, 1H), 2.03-1.95 (m, 2H), 1.82-1.72 (m, 2H), 1.62 (dddd, J=11.6, 4.5, 2.7, 1.4 Hz, 1H), 1.41-1.21 (m, 5H). ¹³C NMR (126 MHz, Chloroform-d) δ 135.32, 132.00, 128.88, 126.71, 46.73, 33.50, 26.21, 25.92.

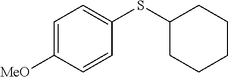 [SMILES: COc1ccc(SC2CCCCC2)cc1]

### Example 54

### Cyclohexyl(4-methoxyphenyl)sulfane (1b)

Following the general procedure using 4-(Methylmercapto)-anisol (77.1 mg, 0.5 mmol) and cyclohexanethiol (145.3 mg, 1.25 mmol). Purification by FC (SiO₂, n-pentane to n-pentane:MTBE 100:1) to afford the title compound (108.1 mg, 98%) as a slightly yellow oil. ¹H NMR (501 MHz, Chloroform-d) δ 7.42-7.35 (m, 2H), 6.89-6.80 (m, 2H), 3.80 (s, 3H), 2.90 (tt, J=10.6, 3.7 Hz, 1H), 1.97-1.89 (m, 2H), 1.79-1.71 (m, 2H), 1.65-1.56 (m, 1H), 1.37-1.15 (m, 5H). ¹³C NMR (126 MHz, Chloroform-d) δ 159.44, 135.71, 125.14, 114.41, 55.44, 48.06, 33.52, 26.26, 25.92.

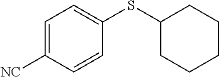 [SMILES: N#Cc1ccc(SC2CCCCC2)cc1]

### Example 55

### 4-(Cyclohexylthio)benzonitrile

Following the general procedure using 4-(Methylthio)-benzonitrile (74.6 mg, 0.5 mmol) and cyclohexanethiol (145.28 mg, 1.25 mmol). Purification by FC (SiO₂, n-pentane to n-pentane:MTBE 20:1) to afford the title compound (64.4 mg, 59%) as a slightly yellow oil. ¹H NMR (501 MHz, Chloroform-d) δ 7.55-7.48 (m, 2H), 7.37-7.30 (m, 2H), 3.29 (tt, J=10.3, 3.7 Hz, 1H), 2.07-1.96 (m, 2H), 1.84-1.76 (m, 2H), 1.66 (dddd, J=12.9, 4.7, 3.0, 1.4 Hz, 1H), 1.50-1.23 (m, 5H). ¹³C NMR (126 MHz, Chloroform-d) δ 144.11, 132.33, 128.71, 119.05, 108.58, 45.06, 33.09, 26.01, 25.75.

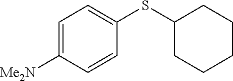 [SMILES: CN(C)c1ccc(SC2CCCCC2)cc1]

### Example 56

### 4-(Cyclohexylthio)-N,N-dimethylaniline

Following the general procedure using N,N-dimethyl-4-(methylthio)aniline (83.6 mg, 0.5 mmol) and cyclohexanethiol (145.28 mg, 1.25 mmol). Purification by FC (SiO₂, n-pentane to n-pentane:MTBE 50:1) to afford the title compound (111.8 mg, 95%) as an orange oil. ¹H NMR (501 MHz, Chloroform-d) δ 7.38-7.30 (m, 2H), 6.69-6.61 (m, 2H), 2.96 (s, 6H), 2.82 (tt, J=10.7, 3.7 Hz, 1H), 2.01-1.89 (m, 2H), 1.74 (dt, J=11.9, 4.1 Hz, 2H), 1.62-1.53 (m, 1H), 1.40-1.14 (m, 5H). ¹³C NMR (126 MHz, Chloroform-d) δ 150.27, 136.18, 119.40, 112.61, 48.35, 40.54, 33.57, 33.39, 26.33, 25.96.

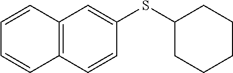 [SMILES: c1ccc2cc(SC3CCCCC3)ccc2c1]

### Example 57

### Cyclohexyl(naphthalen-2-yl)sulfane

Following the general procedure using 2-(Methylthio)-naphthalin (87.1 mg, 0.5 mmol) and cyclohexanethiol (145.3 mg, 1.25 mmol). Purification by FC (SiO₂, n-pentane to n-pentane:MTBE 100:1) to afford the title compound (114.9 mg, 95%) as a colorless oil. ¹H NMR (501 MHz, Chloroform-d) δ 7.86 (d, J=1.7 Hz, 1H), 7.83-7.71 (m, 3H), 7.51-7.36 (m, 3H), 3.24 (tt, J=10.6, 3.7 Hz, 1H), 2.08-1.99 (m, 2H), 1.84-1.74 (m, 2H), 1.63 (dddd, J=12.1, 4.7, 2.9, 1.4 Hz, 1H), 1.49-1.21 (m, 5H). ¹³C NMR (126 MHz, Chloroform-d) δ 133.82, 132.80, 132.22, 130.32, 129.74, 128.34, 127.80, 127.40, 126.53, 125.97, 46.73, 33.52, 29.86, 26.21, 25.93.

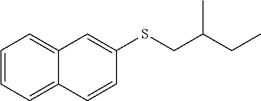 [SMILES: CCC(C)CSc1ccc2ccccc2c1]

### Example 58

### (2-Methylbutyl)(naphthalen-2-yl)sulfane

Following the general procedure using 2-(Methylthio)-naphthalin (87.1 mg, 0.5 mmol) and 2-Methyl-1-butanthiol (130.3 mg, 1.25 mmol). Purification by FC (SiO₂, n-pentane to n-pentane:MTBE 50:1) to afford the title compound (112.9 mg, 98%) as a slightly brown oil. ¹H NMR (501 MHz, Chloroform-d) δ 7.81-7.70 (m, 4H), 7.47 (ddd, J=8.1, 6.7, 1.4 Hz, 1H), 7.46-7.38 (m, 2H), 3.07 (dd, J=12.5, 5.8 Hz, 1H), 2.86 (dd, J=12.5, 7.5 Hz, 1H), 1.79-1.67 (m, 1H), 1.59 (dqd, J=12.9, 7.4, 5.3 Hz, 1H), 1.38-1.25 (m, 1H), 1.07 (d, J=6.7 Hz, 3H), 0.94 (t, J=7.4 Hz, 3H). ¹³C NMR (126 MHz, Chloroform-d) δ 135.25, 133.95, 131.69, 128.36, 127.83, 127.36, 127.08, 126.60, 126.25, 125.53, 40.70, 34.66, 29.86, 29.00, 19.15, 11.44.

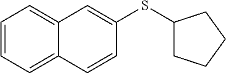 [SMILES: c1ccc2cc(SC3CCCC3)ccc2c1]

### Example 59

### Cyclopentyl(naphthalen-2-yl)sulfane

Following the general procedure using 2-(Methylthio)-naphthalin (87.1 mg, 0.5 mmol) and Cyclopentanthiol (127.8 mg, 1.25 mmol). Purification by FC (SiO₂, n-pentane to n-pentane:MTBE 100:1) to afford the title compound (98.7 mg, 86%) as a slightly yellow oil. ¹H NMR (501 MHz, Chloroform-d) δ 7.82-7.71 (m, 4H), 7.51-7.36 (m, 3H), 3.73 (tt, J=7.2, 6.0 Hz, 1H), 2.17-2.04 (m, 2H), 1.89-1.75 (m, 2H), 1.75-1.58 (m, 3H). ¹³C NMR (126 MHz, Chloroform-d) δ 135.01, 133.91, 131.84, 128.29, 128.20, 127.82, 127.23, 126.55, 125.66, 45.94, 33.72, 29.86, 25.02.

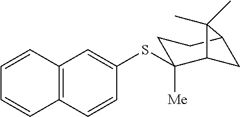 [SMILES: CC1(Sc2ccc3ccccc3c2)CCC2CC1C2(C)C]

### Example 60

### Naphthalen-2-yl((1S,2S,5R)-2,6,6-trimethylbicyclo[3.1.1]heptan-2-yl)sulfane

Following the general procedure using 2-(Methylthio)-naphthalin (87.1 mg, 0.5 mmol) and 2-,3-,10-Mercaptopinane (212.9 mg, 1.25 mmol). Purification by FC (SiO₂, n-pentane to n-pentane:MTBE 100:1) to afford the title compound (134.9 mg, 91%) as a pink oil. ¹H NMR (501 MHz, Chloroform-d) δ 7.78 (dd, J=7.9, 1.3 Hz, 1H), 7.77-7.68 (m, 3H), 7.46 (ddd, J=8.2, 6.8, 1.4 Hz, 1H), 7.42 (ddd, J=8.3, 6.2, 1.6 Hz, 2H), 3.18-3.02 (m, 2H), 2.99-2.90 (m, 0.34H), 2.40-2.23 (m, 2H), 2.15-1.74 (m, 5H), 1.64 (dddd, J=15.1, 10.8, 6.8, 5.6 Hz, 1H), 1.54 (s, 0.20H), 1.51-1.36 (m, 0.37H), 1.29 (s, 0.17H), 1.22 (d, J=11.4 Hz, 3H), 1.06 (s, 2H), 0.88 (d, J=9.7 Hz, 1H), 0.80 (s, 0.52H). ¹³C NMR (126 MHz, Chloroform-d) b 135.30, 134.91, 133.97, 133.95, 131.73, 131.68, 128.38, 128.33, 127.84, 127.43, 127.33, 127.10, 127.08, 126.59, 126.43, 126.11, 125.55, 125.51, 124.77, 45.78, 45.27, 41.42, 41.01, 40.81, 40.69, 40.05, 39.69, 39.59, 38.87, 34.86, 33.46, 29.86, 28.10, 27.71, 26.85, 26.39, 26.30, 24.45, 23.60, 23.47, 22.42, 22.27, 20.25, 19.83.

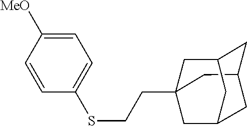 [SMILES: COc1ccc(SCCC23CC4CC(CC(C4)C2)C3)cc1]

### Example 61

### (2-(Adamantan-1-yl)ethyl)(4-methoxyphenyl)sulfane

Following the general procedure using 4-(Methylmercapto)-anisol (77.1 mg, 0.5 mmol) and 2-(1-Adamantyl)-ethanthiol (245.4 mg, 1.25 mmol). Purification by FC (SiO₂, n-pentane to n-pentane:MTBE 50:1) to afford the title compound (146.7 mg, 97%) as a pink oil. ¹H NMR (501 MHz, Chloroform-d) δ 7.35-7.26 (m, 2H), 6.88-6.81 (m, 2H), 3.80 (s, 3H), 2.84-2.76 (m, 2H), 1.94 (p, J=3.1 Hz, 3H), 1.73-1.57 (m, 6H), 1.47 (d, J=2.8 Hz, 6H), 1.41-1.34 (m, 2H). ¹³C NMR (126 MHz, Chloroform-d) δ 158.73, 132.56, 127.27, 114.64, 55.48, 44.11, 42.35, 37.24, 32.88, 29.89, 28.77.

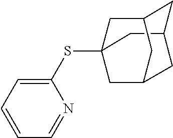 [SMILES: c1ccc(SC23CC4CC(CC(C4)C2)C3)nc1]

### Example 62

### 2-(Adamantan-1-ylthio)pyridine

Following the general procedure using 2-(Methylthio)-pyridin (62.6 mg, 0.5 mmol) and Tricyclo[3.3.1.13,7]decan-1-thiol (210.4 mg, 1.25 mmol). Purification by FC (SiO₂, n-pentane to n-pentane:MTBE 50:1) to afford the title compound (105.7 mg, 96%) as a beige solid. ¹H NMR (501 MHz, Chloroform-d) δ 8.53 (ddd, J=4.9, 2.0, 0.9 Hz, 1H), 7.53 (td, J=7.7, 2.0 Hz, 1H), 7.38 (dt, J=7.9, 1.1 Hz, 1H), 7.10 (ddd, J=7.4, 4.8, 1.1 Hz, 1H), 2.10-2.02 (m, 9H), 1.69 (t, J=3.0 Hz, 6H). ¹³C NMR (126 MHz, Chloroform-d) δ 156.97, 149.73, 136.11, 129.11, 121.44, 50.19, 43.77, 36.43, 30.24.

Ligands Investigation

| | Yield |
|---|---|
| Ligand | (%)^{a,b} |
| 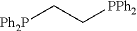 | 46 |
| 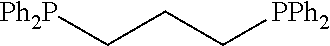 | 51 |
| 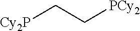 | 88 |
| 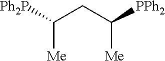 | 75 |
| 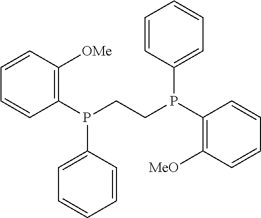 [SMILES: COc1ccccc1P(CCP(c1ccccc1)c1ccccc1OC)c1ccccc1] | 81 |

^{a}Reactions were performed on 0.2 mmol scale of thioanisole.
^{b}Product yield was determined by GC analysis using dodecane as internal standard.

1. A process for a catalytic aryl transfer comprising reacting an aryl-compound (I) with an hydrocarbon (II) in the presence of a Pd- or Ni-catalyst coordinated by electron rich ligands and in the presence of a base in an organic solvent, as represented in the following reaction scheme: 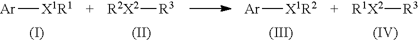
wherein
Ar is aryl, heteroaryl or vinyl, each being optionally substituted by one or more groups selected from straight chain or branched chain alkyl, cycloalkyl, heterocycloalkyl, alkenyl, alkynyl, aryl, aralkyl, heteroaryl, heteroaralkyl, ether, acetal, silyl ether or amine, or by a heterosubstituent;
X¹ and X² may be the same or different and are each S or Se,
R¹ is H or methyl, a straight chain or branched C₂-C₁₆-alkyl or aryl, each optionally being substituted by one or more groups selected from straight chain or branched chain alkyl, cycloalkyl, heterocycloalkyl, alkenyl, alkynyl, aryl, aralkyl, heteroaryl, heteroaralkyl, ether, acetal, silyl ether or amine, or by a heterosubstituent;
R² is a primary, secondary or tertiary alkyl hydrocarbon or aryl hydrocarbon, each being optionally being substituted by one or more groups selected from straight chain or branched chain alkyl, cycloalkyl, heterocycloalkyl, alkenyl, alkynyl, aryl, aralkyl, heteroaryl, heteroaralkyl, or by a heterosubstituent;
R³ is H,
the Pd- or Ni-catalyst coordinated by electron rich ligands is selected from the group consisting of Pd(OAc)₂, Pd₂(dba)₃, PdCl₂, PdCl₂(MeCN)₂, and Ni(COD)₂,
the base is selected from the group consisting of LHMDS, KHMDS, NaHDMS, LiOtBu, KOtBu, and NaOtBu, and
the electron rich ligands may be the same or different and are selected from the group consisting of IPENT, SIPr, Icy and IPr.

2. Process according to claim 1, wherein the catalyst is a Pd-NHC complex.

3. Process according to claim 1, wherein the catalyst is a Ni-bisphosphine complex.

4. Process according to claim 1, wherein the catalyst is present in an amount in the range of 0.05 mol % to 1 mol % of the aryl-compound (I).

5. Process according to claim 1, wherein the base is a lithium base, sodium base or potassium base.

6. Process according to claim 1, wherein the base is present in an amount in the range of 1 equivalent to 6 equivalents of the reaction partners.

7. Process according to claim 1, wherein the aryl-compound (I) is reacted with the hydrocarbon (II) at a temperature in the range of 25° C. to 250° C. for 4 h to 20 h.

8. Process according to claim 1, wherein the aryl-compound (I) is reacted with the hydrocarbon (II) in an aromatic solvent or an aliphatic hydrocarbon solvent.

9. Process according to claim 1, wherein Ar is phenyl, 4-methylphenyl or naphtyl, each optionally being substituted by one or more groups selected from straight chain or branched chain alkyl, ether, acetal, silyl ether or amine.

10. Process according to claim 1, wherein Ar or Ar—X is a component of a polymer.

11. Process according to claim 1, wherein X is S.

12. Process according to claim 1, wherein R1 is H, methyl, phenyl or 4-methoxyphenyl.

13. Process according to claim 1, wherein R² is cyclohexyl, cyclopentyl, 2-methylbutyl, 1-methyl-propyl, nC₁₂H₂₅, adamantyl, 2-phenylethyl, 1-methyl-5-dimethyl-bicyclo[4.1.0]heptyl-, nC₈H₁₇, benzyl, optionally substituted steroid residue, 2-amantadyl-ethyl or C₈H₁₇.

14. Process according to claim 1, wherein the hydrocarbon (II) is present an amount in the range of 0.5 equivalents to 6 equivalents of the aryl-compound (I).

15. Aryl-compound obtainable by the process according to claim 1.
